(19) United States
(12) Patent Application Publication
(10) Pub. No.: US 2022/0054394 A1
(43) Pub. Date: Feb. 24, 2022

(54) GENT FOR DYEING HAIR, CONTAINING AT LEAST ONE ORGANIC SILICON COMPOUND I

(71) Applicant: Henkel AG & Co. KGaA, Duesseldorf (DE)

(72) Inventors: JUERGEN SCHOEPGENS, Schwalmtal (DE); TORSTEN LECHNER, Langenfeld (DE); MARC NOWOTTNY, Moenchengladbach (DE); GABRIELE WESER, Neuss (DE); ULRIKE SCHUMACHER, Duesseldorf (DE); CLAUDIA KOLONKO, Remscheid (DE); CAROLINE KRIENER, Duesseldorf (DE); CARSTEN MATHIASZYK, Essen (DE)

(73) Assignee: Henkel AG & Co. KGaA, Duesseldorf (DE)

(21) Appl. No.: 17/415,547

(22) PCT Filed: Sep. 30, 2019
(86) PCT No.: PCT/EP2019/076466

§ 371 Date: Jun. 17, 2021

(30) Foreign Application Priority Data
Dec. 19, 2018 (DE) 10 2018 132 893.1

(51) Int. Cl. A61K 8/898; A61Q 5/10; A61K 8/891; A61K 8/81

(52) U.S. Cl. CPC A61K 8/898; A61Q 5/10; A61K 2800/884; A61K 8/8147; A61K 2800/43; A61K 8/891

(57) ABSTRACT

The subject of the present application is an agent for coloring keratinous material, in particular human hair, which contains in a cosmetic carrier (a) at least one special organic silicon compound, and (b) at least one coloring compound. The present disclosure further relates to a multicomponent packaging unit (kit-of-parts) for dyeing keratinous material, in particular human hair, which comprises, packaged separately from one another in three different containers, agents (I), (II) and (III), agent (I) containing the at least one organic silicon compound (a) and the at least one color-imparting compound (b) being contained either in agent (II) together with water and/or in agent (III) together with at least one film-forming, hydrophilic polymer (c). Finally, the present disclosure also relates to processes for dyeing keratinous material using the agents described, and to processes for preparing the organic silicon compound (a).

### CROSS-REFERENCE TO RELATED APPLICATION

This application is a U.S. National-Stage entry under 35 U.S.C. § 371 based on International Application No. PCT/EP2019/076466, filed Sep. 30, 2019, which was published under PCT Article 21(2) and which claims priority to Germany Application No. 10 2018 132 893.1, filed Dec. 19, 2018, which are all hereby incorporated in their entirety by reference.

### TECHNICAL FIELD

The subject of the present application is an agent for coloring keratinous material, in particular human hair, which contains in a cosmetic carrier (a) at least one special organic silicon compound, and (b) at least one coloring compound. The present disclosure further relates to a multicomponent packaging unit (kit-of-parts) for dyeing keratinous material, in particular human hair, which comprises, packaged separately from one another in three different containers, agents (I), (II) and (III), agent (I) containing the at least one organic silicon compound (a) and the at least one color-imparting compound (b) being contained either in agent (II) together with water and/or in agent (III) together with at least one film-forming, hydrophilic polymer (c). Finally, the present disclosure also relates to processes for dyeing keratinous material using the agents described, and to processes for preparing the organic silicon compound (a).

### BACKGROUND

The change in shape and color of keratin fibers, especially hair, is an important area of modern cosmetics. To change the hair color, the expert knows various coloring systems depending on coloring requirements. Oxidation dyes are usually used for permanent, intensive dyeing's with good fastness properties and good grey coverage. Such colorants usually contain oxidation dye precursors, so-called developer components and coupler components, which form the actual dyes under the influence of oxidizing agents such as hydrogen peroxide. Oxidation dyes are exemplified by very long-lasting dyeing results.

When direct dyes are used, ready-made dyes diffuse from the colorant into the hair fiber. Compared to oxidative hair dyeing, dyeing's obtained with direct dyes have lower durability and faster wash-out. Dying' s with direct dyes usually remain on the hair for a period of between about 5 and about 20 washes.

For short-term color changes on the hair and/or skin, the use of color pigments is known. Color pigments are generally understood to be insoluble, coloring substances. These are present undissolved in the form of small particles in the coloring formulation and are merely deposited externally on the hair fibers and/or skin surface. Therefore, they can usually be removed again without residue by a few washes with detergents containing surfactants. Various products of this type are available on the market under the name hair mascara. If the user wants particularly long-lasting dyeing's, the use of oxidative dyes has so far been his only option. However, despite multiple optimization attempts, an unpleasant ammonia odor or amine odor cannot be completely avoided during oxidative hair coloring. The hair damage still associated with the use of oxidative dyes also has a negative effect on the user's hair.

EP 2168633 B1 deals with the task of producing long-lasting hair colorations using pigments. The paper teaches that when a combination of pigment, organic silicon compound, hydrophobic polymer and a solvent is used on hair, it is possible to produce colorations that are particularly resistant to shampooing.

During the revision of the doctrine of EP 2168633 B1, its formulations have been adjusted. It has been shown that one disadvantage of these formulations is their poor storage stability. With the pigments and the hydrophobic polymers, the formulations contain very poorly soluble substances, which could be brought into dispersion for a short time when the formulations were prepared, but over longer storage periods agglomerated, settled, or separated from the water phase. Depending on the chosen application concentration of pigment and hydrophobic polymer, it has also proved difficult to get the poorly soluble substances sufficiently finely dispersed in dispersion directly during production.

### BREIF SUMMARY

Compositions, multicomponent packaging units (kits-of-parts), and methods for dyeing keratinous material, in particular human hair, are provided herein. In an exemplary embodiment, the composition comprises, in a cosmetic carrier, (a) at least one organic silicon compound obtainable by partial condensation under reduced pressure of at least one amino silane (a1) of the formula (I), R₁R₂N-L-Si(OR₃)a(R₄)₃₋ₐ (I), wherein R1, R2 independently represent a hydrogen atom, a C₁-C₆ alkyl group, a hydroxy-C₁-C₆ alkyl group, a C₂-C₆ alkenyl group, an amino-C₁-C₆ alkyl group, an amino-C₁-C₆ alkyl-amino-C₁-C₆ alkyl group or a group of formula (III), -(L′)_{c}-Si(R₆)_{b}(OR₅)_{3-b} (III), wherein L and L′ each independently represent a linear or branched C₁-C₂₀ divalent alkylene group, R₃ and R₅ independently represent a hydrogen atom or a C₁-C₆ alkyl group, R₄ and R₆ independently represent a C₁-C₆ alkyl or a C₂-C₆ alkenyl group, a and b each independently represent an integer from 2 to 3, and c is 0 or 1; and optionally at least one second silane (a2) of formula (II), R₇—Si(OR₈)d(R₉)_{3-d} (II), wherein R₇ represents a linear or branched C₁-C₁₂ alkyl group, hydroxy-C₁-C₁₂ alkyl group or C₂-C₁₂ alkenyl group, R₈ represents a hydrogen atom or a C₁-C₆ alkyl group, R₉ is a C₁-C₆ alkyl group or a C₂-C₆ alkenyl group, d is an integer from 2 to 3; and (b) at least one colorant compound.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The purpose of the present disclosure was to provide a dyeing system with fastness properties comparable to those of oxidative dyeing. In particular, the wash fastness should be outstanding, but the use of the oxidation dye precursors normally used for this purpose should be avoided. A technology was sought that would make it possible to fix the coloring compounds known from state-of-the-art technology (such as pigments and direct dyes) to the hair in an extremely permanent manner Here, a sufficiently high storage stability of the formulations should be ensured. In addition, the production process of the formulations should also be simplified or optimized.

Surprisingly, it has now been found that the problem can be excellently solved if keratinous materials, in particular human hair, are colored with an agent which contains in a cosmetic carrier at least one specific organic silicon compound and at least one coloring compound.

A first object of the present disclosure is therefore an agent for coloring keratinous material, in particular human hair, containing
(a) at least one organic silicon compound obtainable by partial condensation under reduced pressure of at least one amino silane (a1) of the formula (I),

R₁R₂N-L-Si(OR₃)ₐ(R₄)₃₋ₐ (I),

where

R₁, R₂ independently represent a hydrogen atom, a C₁-C₆ alkyl group, a hydroxy-C₁-C₆ alkyl group, a C₂-C₆ alkenyl group, an amino-C₁-C₆ alkyl group, an amino-C₁-C₆ alkyl-amino-C₁-C₆ alkyl group or a group of formula (III),

-(L′)_{c}-Si(OR₅)_{b}(R₆)_{3-b} (III),

where

L represents a linear or branched divalent alkylene group, arylene group, saturated cycloaliphatic group, arylenealkylene group, alkylenearylene group, alkylenearylenealkylene group or arylenealkylenearylene group, each having up to about 30 carbon atoms, or a hetero-derivative thereof in which about 1 to about 4 carbon atoms are replaced by O, S or NR₁, preferably a linear or branched C₁₋₆ alkylene group, more preferably a linear C₁₋, C₂ or C₃ alkylene group,

L′ represents a linear or branched divalent alkylene group, arylene group, saturated cycloaliphatic group, arylenealkylene group, alkylenearylene group, alkylenearylenealkylene group or arylenealkylenearylene group, each having up to about 30 carbon atoms, or a hetero-derivative thereof in which about 1 to about 4 carbon atoms are replaced by O, S or NH, preferably a linear or branched C₁₋₆ alkylene group, more preferably a linear C₁₋, C₂ or C₃ alkylene group,

R₃ and R₅ independently represent a hydrogen atom or a C₁-C₆ alkyl group, preferably a C₁-C₂ alkyl group,

R₄ and R₆ independently represent a C₁-C₆ alkyl or a C₂-C₆ alkenyl group,

a and b each independently represent an integer from about 2 to about 3, preferably about 3, and

c is 0 or 1;

and optionally at least one second silane (a2) of formula (II)

R₇—Si(OR₈)_{d}(R₉)_{3-d} (II),

where

R₇ represents a linear or branched C₁-C₁₂ alkyl group, C₁-C₁₂ alkoxy group, hydroxy C₁-C₁₂ alkyl group or C₂-C₁₂ alkenyl group, preferably a linear C₁-C₆ alkyl, C₁-C₆ alkoxy or C₂-C₆ alkenyl group, more preferably C₁-C₂ alkyl group,

R₈ represents a hydrogen atom or a C₁-C₆ alkyl group, preferably a C₁-C₂ alkyl group,

R₉ is a C₁-C₆ alkyl or a C₂-C₆ alkenyl group,

d is an integer from about 2 to about 3; and
(b) at least one colorant compound, preferably from the group of photochromic dyes, thermochromic dyes, pigments and/or direct dyes.

In the course of the work leading to the present disclosure, it was found that the use of the organic silicon compounds (a) as contemplated herein resulted in formulations with excellent storage stability. Surprisingly, it was further found that a very resistant film could be produced on the keratinous material using the organic silicon compounds (a) as contemplated herein. For this reason, extremely washfast stains with good resistance to shampooing were obtained on the keratinous material.

### Agent for Dyeing Keratinous Material

Keratinous material includes hair, skin, nails (such as fingernails and/or toenails). Wool, furs, and feathers also fall under the definition of keratinous material.

Preferably, keratinous material is understood to be human hair, human skin, and human nails, especially fingernails and toenails. Keratinous material is understood to be human hair.

In various embodiments, the compositions as contemplated herein contain the compounds (a) and (b) essential to the present disclosure in a cosmetic carrier.

The compositions as contemplated herein may preferably contain the compounds (a) and (b) essential to the present disclosure in separate containers. The formulations containing compound (a) or (b) in these containers may each be formulated differently, i.e. may also contain different cosmetic carriers. The formulations contained in the various containers can then be mixed or combined before or during use. Alternatively, only one of the two compounds may be present pre-formulated with a cosmetic carrier and the other is combined, if at all necessary, with a suitable carrier or the other compound pre-formulated with the carrier only before use. In still another alternative, the two compounds are mixed with a cosmetic carrier only at the time of application, either individually or together.

The term “compositions for coloring keratinous material” as used herein thus refers both to compositions in which components (a) and (b) are contained spatially separated from each other and to the ready-to-use compositions in which components (a) and (b) can be used both in admixture with each other and optionally other components or further separately or each in admixture with other components. Specific embodiments for such elements in the form of kits and their use or methods of use are described in detail herein.

The carriers used in the application are, for example, suitable aqueous or aqueous-alcoholic carriers. To hair coloring, such carriers are, for example, creams, emulsions, gels, or also surfactant-containing foaming solutions, such as shampoos, foam aerosols, foam formulations or other preparations suitable for application to the hair.

The cosmetic carrier preferably contains water, which means that the carrier contains at least about 2% by weight of water based on its weight. Preferably, the water content is above about 5 wt. %, further preferably above about 10 wt. % still further preferably above about 15 wt. %. The cosmetic carrier can also be aqueous alcoholic. For the purposes of the present disclosure, aqueous alcoholic solutions are understood to mean aqueous solutions containing about 2 to about 70% by weight of a C₁-C₄ alcohol, in particular ethanol or isopropanol. The agents as contemplated herein may additionally contain other organic solvents, such as methoxybutanol, benzyl alcohol, ethyl diglycol or 1,2-propylene glycol. Preferred are all water-soluble organic solvents.

It goes without saying that the compounds (a) are not prepared, stored and/or transported pre-formulated in an aqueous or water-containing carrier, but contact with water, for example in the form of a carrier, takes place only shortly before or during use to avoid premature crosslinking/condensation of the silanes. Accordingly, the foregoing discussion of carriers refers particularly to such carriers as are used in the use of the agents, and not to the carriers in which the components of the agents are stored and transported.

The term “coloring agent” is used in the context of the present disclosure to refer to a coloring of the keratin material, of the hair, caused using coloring compounds, such as thermochromic and photochromic dyes, pigments, mica and/or direct dyes. In this staining process, the colorant compounds are deposited in a particularly homogeneous and smooth film on the surface of the keratin material or diffuse into the keratin fiber. The film forms in situ by oligomerization or polymerization of the organic silicon compound(s), and by the interaction of the color-imparting compound and organic silicon compound and optionally other ingredients, such as a film-forming hydrophilic polymer.

### Organic Silicon Compounds

As the first constituent (a) essential to the present disclosure, the compositions as contemplated herein comprise at least one organic silicon compound obtainable by partial condensation under reduced pressure of at least one amino silane (a1) of the formula (I),

R₁R₂N-L-Si(OR₃)ₐ(R₄)₃₋ₐ (I),

where

R₁, R₂ independently represent a hydrogen atom, a C₁-C₆ alkyl group, a hydroxy-C₁-C₆ alkyl group, a C₂-C₆ alkenyl group, an amino-C₁-C₆ alkyl group, an amino-C₁-C₆ alkyl-amino-C₁-C₆ alkyl group or a group of formula (III),

-(L′)_{c}-Si(OR₅)_{b}(R₆)_{3-b} (III),

where

L represents a linear or branched divalent alkylene group, arylene group, saturated cycloaliphatic group, arylenealkylene group, alkylenearylene group, alkylenearylenealkylene group or arylenealkylenearylene group, each having up to about 30 carbon atoms, or a hetero-derivative thereof in which about 1 to about 4 carbon atoms are replaced by O, S or NR₁, preferably a linear or branched C₁₋₆ alkylene group, more preferably a linear C₁₋, C₂ or C₃ alkylene group,

L′ represents a linear or branched divalent alkylene group, arylene group, saturated cycloaliphatic group, arylenealkylene group, alkylenearylene group, alkylenearylenealkylene group or arylenealkylenearylene group, each having up to about 30 carbon atoms, or a hetero-derivative thereof in which about 1 to about 4 carbon atoms are replaced by O, S or NH, preferably a linear or branched C₁₋₆ alkylene group, more preferably a linear C₁₋, C₂ or C₃ alkylene group,

R₃ and R₅ independently represent a hydrogen atom or a C₁-C₆ alkyl group, preferably a C₁-C₂ alkyl group,

R₄ and R₆ independently represent a C₁-C₆ alkyl or a C₂-C₆ alkenyl group,

a and b each independently represent an integer from about 2 to about 3, preferably about 3, and

Is c 0 or 1;

and optionally at least one second silane (a2) of formula (II)

R₇—Si(OR₈)_{d}(R₉)_{3-d} (II),

where

R₇ represents a linear or branched C₁-C₁₂ alkyl group, C₁-C₁₂ alkoxy group, hydroxy C₁-C₁₂ alkyl group or C₂-C₁₂ alkenyl group, preferably a linear C₁-C₆ alkyl, C₁-C₆ alkoxy or C₂-C₆ alkenyl group, more preferably C₁-C₂ alkyl group,

R₈ represents a hydrogen atom or a C₁-C₆ alkyl group, preferably a C₁-C₂ alkyl group,

R₉ is a C₁-C₆ alkyl or a C₂-C₆ alkenyl group,

d stands for an integer from about 2 to about 3.

Organic silicon compounds, alternatively referred to as organosilicon compounds, are compounds that either have a direct silicon-carbon (Si—C) bond or in which the carbon is attached to the silicon atom via an oxygen nitrogen or sulfur atom. The organic silicon compounds of the present disclosure are compounds containing at least two silicon atoms, preferably three or more.

The organic silicon compounds (a) as contemplated herein are obtainable by partial condensation of the silanes described herein and are also referred to herein as “precondensates” or “partial condensates”, since they usually include at least two monomeric units of formula (I) or at least one monomeric unit of formula (I) and one monomeric unit of formula (II), which have been combined by employing a condensation reaction. The term “partial ” or “precondensate” further refers to the fact that the organic silicon compounds (a) each still comprise one or more hydroxyl groups or hydrolysable groups per molecule, i.e., are only partially condensed so that they are still further condensable/crosslinkable when used for coloring. The hydrolysable group(s) is (are) preferably a C₁-C₆ alkoxy group, especially an ethoxy group or a methoxy group. It is preferred when the hydrolysable group is directly bonded to the silicon atom. For example, if the hydrolysable group is an ethoxy group, the organic silicon compound preferably contains a structural unit R′R″R′″Si—O—CH₂—CH₃. The radicals R′, R″ and R′″ represent the three remaining free valences of the silicon atom.

It is preferred that the organic silicon compounds (a) contain at least about 3 or about 4 groups amino groups of the formulae —NH, —NH₂ or —NR₁R₂. This ensures that sufficient adhesion is obtained on the surface of the keratinous materials.

Examples of a C₁-C₆ alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino-C₁-C₆-alkyl group and an amino-C₁-C₆-alkyl-amino-C₁-C₆-alkyl group (also N-(aminoalkyl)-aminoalkyl-) are groups of the formula —(CH₂)ₒ—NH₂ and —(CH₂)ₒ—NH—(CH₂)ₚ—NH₂, where o and p are each 1, 2, 3, 4, 5 or 6, preferably 2 or 3, more preferably 2. Particularly preferred are the aminomethyl group, the 2-aminoethyl group (NH₂—(CH₂)₂—), the 3-aminopropyl group (NH₂—(CH₂)₃—), and the 2-aminoethyl-2-aminoethyl group (NH₂—(CH₂)₂—NH—(CH₂)₂—). The 2-aminoethyl group as well as the 2-aminoethyl-2-aminoethyl group (NH₂—(CH₂)₂—NH—(CH₂)₂—) are particularly preferred.

In various embodiments, the divalent groups for which L and L′ represent each comprise up to about 20 carbon atoms, preferably up to about 20 carbon atoms, more preferably up to about 12 carbon atoms, for example 1, 2, 3, 4, 5, 6, 7, 8, 9, 10, 11 or 12 carbon atoms.

Examples of a linear divalent C₁-C₃₀ alkylene group include, but are not limited to, the methylene group (—CH₂—), the ethylene group (—CH₂—CH₂—), the n-propylene group (—CH₂—CH₂—CH₂—), and the n-butylene group (—CH₂—CH₂—CH₂—CH₂—). The propylene group (—CH₂—CH₂—CH₂—) is particularly preferred. From a chain length of 3 C atoms, divalent alkylene groups can also be branched. Examples of branched C₃-C₃₀-divalent alkylene groups are (—CH₂—CH(CH₃)—), (—CH₂—CH(CH₃)—CH₂—), (—CH₂—CH(CH₃)—CH₂—CH₂—), and (—CH₂—C(CH₃)₂—CH₂—CH₂—).

Examples of a divalent arylene group include, but are not limited to, 1,4-phenylene, 1,3-phenylene, and 1,2-phenylene (each —C₆H₄—).

Examples of saturated cycloaliphatic groups include 1,4-cyclohexylene and 1,3-cyclopentylene.

Arylene alkylene groups, alkylene arylene groups, alkylene arylene alkylene groups and arylene alkylene arylene groups are obtained by combining the alkylene and arylene groups described above. Examples include, but are not limited to, ethylene-p-phenylene (—CH₂—CH₂—C₆H₄—), p-phenylene-ethylene (—C₆H₄—CH₂—CH₂—), ethylene-p-phenylene-ethylene (—CH₂—CH₂—C₆H₄—CH₂—CH₂—), and p-phenylene-ethylene-p-phenylene (—C₆H₄—CH₂—CH₂—C₆H₄—).

Hetero-derivatives of the above-described groups include, but are not limited to, ethylaminoethyl, ethylaminopropyl, ethyl-3-aminoisobutyl, propylaminopropyl, hexylaminomethyl, ethylaminoundecyl, oxypropylaminopropyl, ethyl-2-aminoethyl-3-aminopropyl, ethylthiopropyl, ethoxypropyl, and ethoxyethyl. It is preferred that in the hetero derivatives of said groups about 1 or about 2 carbon atoms, preferably only about 1 carbon atom is exchanged for O, S or NR₁. In various preferred embodiments, NR₁ in these hetero-derivatives is preferably NH or N—C₁₋₆ alkyl.

The organic silicon compounds (a) are preferably obtainable by partial condensation of amino silanes (a1) of the formula (I).

In various embodiments, R₁ and R₂ independently represent a hydrogen atom or a C₁-C₆ alkyl group, particularly methyl or ethyl. Particularly preferably, at least one of R₁ and R₂ most preferably both represents a hydrogen atom. If only one of the two radicals stands for a hydrogen atom, the other preferably stands for a C₁-C₆ alkyl group or an amino-C₁-C₆ alkyl group/amino-C₁-C₆ alkyl group, particularly preferably methyl, ethyl, 2-aminoethyl or 2-aminoethyl-2-aminoethyl.

Also included are variants in which one of the radicals R¹ and R² represents a group of the formula (III). Those in which L′=L and R₆═R₄, R₅═R₃ and b=a, i.e., those which carry symmetrical silane groups on the nitrogen atom, are preferred. In such embodiments, R₁ preferably represents a hydrogen atom, a C₁-C₆ alkyl group, a hydroxy-C₁-C₆ alkyl group, is a C₂-C₆ alkenyl group or an amino-C₁-C₆ alkyl group and R₂ is a grouping of formula (III) wherein b, L′, R₅ and R₆ are preferably identical to a, L, R³ and R⁴.

In the middle part of the organic silicon compound is the structural unit or linker -L- which preferably stands for a linear or branched, divalent C₁-C₂₀ alkylene group. Particularly preferably, -L- represents a linear, divalent C₁-C₂₀ alkylene group. Further preferably -L- stands for a linear divalent C₁-C₆ alkylene group. Particularly preferred -L stands for a methylene group (CH₂—), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—) Also preferred are branched divalent C₃-C₂₀ alkylene groups, especially dimethylbutyl (3,3-dimethylbutyl).

In another preferred embodiment, R₁, R₂ both represent a hydrogen atom and L represents a linear divalent C₁-C₆ alkylene group, preferably a propylene group (—CH₂—CH₂—CH₂—) or an ethylene group (—CH₂—CH₂—).

In the terminal structural unit —Si(OR₃)ₐ(R₄)₃₋ₐ, R₃ is hydrogen or C₁-C₆ alkyl, and R₄ is C₁-C₆ alkyl or C₂₋₆ alkenyl. Particularly preferably, R₃ stands for a C₁-C₆ alkyl group, especially a methyl group or an ethyl group. a is preferably about 3. If a is about 2, then R₄ is preferably methyl, ethyl, or vinyl.

It is preferred in various embodiments that R₃ and R₅, if present, do not represent a hydrogen atom.

Organic silicon compounds of the formula (I) which are particularly suitable for solving the problem as contemplated herein are
(3-Aminopropyl)triethoxysilane (3-Aminopropyl)trimethoxysilae 1-(3-Aminopropyl)silanetriol
(2-Aminoethyl)triethoxysilane
(2-Aminoethyl) trimethoxysilane
1-(2-Aminoethyl)silanetriol,
Aminomethyltrimethoxysilane
Aminomethyltriethoxysilane
Aminomethylsilanetriol
N-(2-aminoethyl)-3-Aminopropyltrimethoxysilane
N-(2-aminoethyl)-3-Aminopropyltriethoxysilane
N-(2-aminoethyl)-3-Aminopropylsilanetriol
(3-Dimethylaminopropyl) triethoxysilane
(3-Dimethylaminopropyl) trimethoxysilane
1-(3-Dimethylaminopropyl) silanetriol
(2-Dimethylaminoethyl)triethoxysilane
(2-Dimethylaminoethyl)trimethoxysilane
1-(2-Dimethylaminoethyl)silanetriol
Dimethylaminomethyltrimethoxysilane
Dimethylaminomethyltriethoxysilane
Dimethylaminomethylsilanetriol
(3-Diethylaminopropyl)triethoxysilane
(3-Dimethylaminopropyl)trimethoxysilane
1-(3-Diethylaminopropyl)silanetriol
(2-Diethylaminoethyl)triethoxysilane
(2-Diethylaminoethyl)trimethoxysilane
1-(2-Diethylaminoethyl)silanetriol,
Diethylaminomethyltrimethoxysilane,
Diethylaminomethyltriethoxysilane,
Diethylaminomethylsilanetriol
3-(Trimethoxysilyl)-N-[3-(trimethoxysilyl)propyl]-1-propanamine
3-(Triethoxysilyl)-N-[3-(triethoxysilyepropyl]-1-propanamine
N-Methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]ethanol
2-[Bis[3-(triethoxysilyl)propyl]amino]ethanol
3-(Trimethoxysilyl)-N,N-bis[3-(trimethoxysilyl)propyl]-1-Propanamine
3-(triethoxysilyl)-N,N-bis[3-(triethoxysilyl)propyl]-1-Propanamine
N1,N1-Bis[3-(trimethoxysilyl)propyl]-1,2-Ethanediamine
N1,N1-Bis[3-(triethoxysilyl)propyl]-1,2-Ethanediamine
N,N-Bis[3-(trimethoxysilyl)propyl]-2-Propen-1-amine, and
N,N-Bis[3-(triethoxysilyl)propyl]-2-Propen-1-amine

The alkoxysilanes are generally preferred here over the silanols. The amino silanes are commercially available. (3-aminopropyl)trimethoxysilane, for example, can be purchased from Sigma-Aldrich. Also (3-aminopropyl)triethoxysilane is commercially available from Sigma-Aldrich. Bis (trimethoxysilylpropyl) amine with the CAS number 82985-35-1 can be purchased from Sigma-Aldrich, for example. Bis[3-(triethoxysilyl)propyl]amines with CAS number 13497-18-2 can be purchased from Sigma-Aldrich, for example. N-Methyl-3-(trimethoxysilyl)-N-[3-(trimethoxysilyl)propyl]-1-propanamine is alternatively known as bis(3-trimethoxysilylpropyl)-N-methylamine and can be purchased commercially from Sigma-Aldrich or Fluorochem. 3-(Triethoxysilyl)-N,N-bis[3-(triethoxysilyl)propyl]-1-propanamine with CAS number 18784-74-2 can be purchased from Fluorochem or Sigma-Aldrich, for example.

In various embodiments of the present disclosure in which in the amino silanes (a1) of formula (I) R₁ or R₂ is a group of formula (III), no silane (a2) of formula (II) is used. In contrast, in embodiments in which amino silanes are used in which neither R₁ nor R₂ is a group of formula (III), a silane (a2) of formula (II) is preferably used.

In the silanes (a2) of formula (II), R₇ represents a linear or branched C₁-C₁₂ alkyl group, C₁-C₁₂ alkoxy group, hydroxy C₁-C₁₂ alkyl group or C₂-C₁₂ alkenyl group, preferably a linear C₁-C₆ alkyl, C₁-C₆ alkoxy or C₂-C₆ alkenyl group, such as Methyl, Ethyl, Propyl, Butyl, Methoxy, Ethoxy and Vinyl, more preferably C₁-C₂ Alkyl group, i.e., Methyl or Ethyl.

R₈ represents a hydrogen atom or a C₁-C₆ alkyl group, preferably a C₁-C₆ alkyl group, methyl and ethyl being particularly preferred. It may be preferred in various embodiments that no R₈ is hydrogen.

R₉ represents a C₁-C₆ alkyl or a C₂-C₆ alkenyl group, in particular Methyl, Ethyl, or Vinyl.

In the compounds of formula (II), d is preferably about 3. This is also particularly preferred when R₇ is an Alkoxy group. Particularly preferably, R₈ stands for a C₁-C₆ Alkyl group, especially a Methyl group or an Ethyl group. When b is about 2, R₉ is preferably Methyl, Ethyl, or Vinyl, more preferably Methyl or Ethyl, even more preferably Methyl.

In various embodiments, the Silane (a2) comprises a compound of formula (II) wherein d is about 3 and R₇ and Rs are each Methyl or Ethyl.

In various embodiments, a composition as contemplated herein is exemplified in that, for the preparation of the at least one organic Silicon compound (a), in addition to the Amino silane of formula (I), a Silane of formula (II) is also used, the latter preferably being selected from the group of Methyltrimethoxysilane; Ethyltrimethoxysilane; Methyltriethoxysilane; Ethyltriethoxysilane; Dimethyldimethoxysilane; Diethyldiethoxysilane; Dimethyldiethoxysilane; Diethyldimethoxysilane; Vinyltrimethoxysilane; Vinyltriethoxysilane; Tetraethylorthosilicate; and Tetramethylorthosilicate.

In particular, the Trialkoxysilanes mentioned are preferred.

In various embodiments, multiple Amino silanes (a1) of formula (I) and/or multiple Silanes (a2) of formula (II) are used.

In various embodiments in which Amino silanes (a1) of the formula (I) and Silanes (a2) of the formula (II) are used, the mass ratio of all compounds of the formula (I) to all compounds of the formula (II) is preferably from about 5:1 to about 1:20, more preferably from about 1:1 to about 1:10, still more preferably from about 1:2 to about 1:5.

In various embodiments in which amino silanes (a1) of the formula (I) and silanes (a2) of the formula (II) are used, the molar ratio of all compounds of the formula (I) to all compounds of the formula (II) is preferably from about 2:1 to about 1:30, more preferably from about 1:1 to about 1:20, still more preferably from about 1:2 to about 1:10.

The organic Silicon compounds (a) are generated by a condensation reaction of the Amino silanes (a1) with each other or with the Silanes (a2). The condensation is a partial condensation, where “Partial condensation” in this context means that not all condensable groups react with each other, so that the resulting organic silicon compound still has on average at least one hydrolysable/condensable group per molecule. Preferably, the average number of condensable/hydrolysable groups per molecule is at least about 1.5, more preferably at least about 2. In various embodiments, the quotient of the molar ratio of Si/hydrolysable group (especially alkoxy group) in the produced molecule/oligomer is at least about 0.3, preferably at least about 0.5, more preferably at least about 0.7, for example at least about 1.

Organic silicon compounds a) as contemplated herein have, for example, a hydrolysable group/alkoxy group content of, for example, about 5 to about 30% by weight.

Organic silicon compounds a) as contemplated herein can be chain or cyclic. They are mixtures of chain and/or cyclic oligomers.

The degree of oligomerization is typically about 2 to about 30, i.e., the organic silicon compounds a) as contemplated herein include about 2 to about 30 monomeric units derived from the compounds a1 and, if used, also a2, preferably from about 3 to about 20 units.

The number of remaining condensable/hydrolysable groups can be controlled by the hydrolysis conditions. Preferably, the amino silanes (a1) and the silanes (a2), which are preferably alkoxysilanes, methoxy- or ethoxy silanes, are hydrolyzed with water in a first step. In this process, the water is used in a sub-stoichiometric quantity, i.e., a quantity that is less than the quantity that would theoretically be would be required to hydrolyze all hydrolysable groups present on the Si atoms, i.e., the alkoxysilane groups. Preferably, the amount of water used for this purpose is at least about 10% below the amount stoichiometrically required for complete hydrolysis, and preferably at least about 20% below. Particularly preferably, the amount of water used for hydrolysis is about 0.2 to about 2.5 mol of water per 1 mol of Si, preferably from about 0.4 to about 2.0 mol of water per 1 mol of Si, more preferably from about 0.6 to about 1.6 mol or about 0.7 to about 1.6 mol or about 0.8 to about 1.3 mol or about 0.8 to about 1.2 mol or about 0.85 to about 1.0 mol of water per 1 mol of Si. During hydrolysis, the hydrolysable groups on the Si atoms are hydrolyzed and, if they are alkoxy groups, the corresponding alcohols are split off. Si—OH groups remain on the silicon, which can react with each other in the next step in a condensation reaction, i.e., with water splitting off.

The water can be added for the hydrolysis reaction continuously, in partial amounts or directly as a total amount. The addition is preferably made to the silanes (a1) and, if necessary, (a2) presented, with stirring. To ensure temperature control, the reaction vessel can be cooled, or the amount and rate of water added can be adjusted. Depending on the amount of silanes used, the addition and reaction can take place over a period of about 2 minutes to about 72 hours. In preferred embodiments, the water is added continuously.

It may be preferred in various embodiments that the temperature during hydrolysis does not exceed about 75° C., preferably about 60° C., more preferably about 50° C. Preferably, the temperature during the hydrolysis reaction and optionally also during the subsequent condensation reaction is in the range of about 10 to about 75° C., preferably from about 20 to about 60° C.

It is further preferred that the hydrolysis reaction takes place under inert gas, such as nitrogen, or it is otherwise ensured that the reaction mixture does not meet additional moisture, such as humidity. The reaction therefore preferably takes place in a reaction vessel that is closed to the ambient atmosphere or under the exclusion of moisture.

The hydrolysis reaction is followed by a condensation reaction in which the Si—O—Si bonds are formed. In practice, the rate of reaction is fast enough that hydrolysis and condensation (precondensation) occur almost in parallel. However, to shift the equilibrium of the condensation reaction in the direction of the products, it is provided as contemplated herein that the condensation reaction (partial condensation) is carried out under reduced pressure to remove the resulting alcohols (in the case of alkoxysilanes used) and possibly also any water formed from the reaction mixture by distillation and to transfer them to the gas phase. This suppresses the back reaction and shifts the equilibrium of the reaction to the side of the condensates. Preferably, the reduced pressure is achieved by a vacuum distillation in which the reaction mixture is reduced pressure, typically to a maximum of about 800 mbar, preferably to a maximum of about 500 mbar, for example about 50-800 or about 50-500 mbar, and the volatile alcohols and possibly also water are condensed and collected as liquid distillate in a receiver. Distillation can optionally be carried out with cooling of the volatile alcohols/water by employing a cooler. The reduced pressure can be generated by employing common processes known in the prior art, typically with a vacuum pump.

In various embodiments of the present disclosure, silanes (a1) and (a2) are predominantly, i.e., at least about 50% by weight, preferably at least about 75% by weight, almost exclusively, i.e., at least about 90% by weight, preferably about 95% by weight, or exclusively those which carry methoxy silane or ethoxy silane groups, di- and trimethoxy- and ethoxy silanes, particularly preferably trimethoxy- or triethoxysilane. These have the advantage that methanol and ethanol are released during hydrolysis and condensation, respectively, which can be easily removed from the reaction mixture by vacuum distillation due to their boiling points.

In various embodiments of the present disclosure, the preparation is carried out in a two- or multi-step process in which, in a first step, hydrolysis is carried out by adding sub-stoichiometric amounts of water, either continuously, stepwise or in one, under normal pressure. Only after the water has been added, preferably completely, is reduced pressure applied in a subsequent step and the resulting/emerging alcohols removed by vacuum distillation. In this embodiment, vacuum distillation is preferably performed after at least about 50% by weight of the total designed amount of water, preferably at least about 70%, about 80%, about 90%, about 95% or about 100% by weight of the water has been added, preferably continuously.

Alternatively, in various embodiments of the present disclosure, vacuum distillation may be performed simultaneously with hydrolysis. In such embodiments, the pressure is already reduced before the water is added, at the start of the addition, or after about 5-20 wt % of the total planned amount of water has been added.

The condensation reaction at reduced pressure can be carried out at elevated temperature. For this purpose, the reaction vessel can be actively heated. In various embodiments, the temperature can be adjusted such that the alcohols released during the condensation reaction can be vaporized and removed at the applied reduced pressure. In various preferred embodiments, however, the temperature is no more than about 75° C., preferably no more than about 60° C. It may be further preferred that there is no active heating of the reaction mixture and that any increase in temperature above ambient temperature is caused only by the exotherm of the hydrolysis.

In various embodiments of the present disclosure, the reaction may also be carried out in the presence of solvents, i.e., alcohols such as methanol or ethanol. These are then usually used in about 0.1 to about 5 times the amount by weight based on the silanes used and then removed by distillation.

In various embodiments, the reaction under reduced pressure yields a product containing less than about 5 wt %, preferably less than about 2 wt %, more preferably less than about 1 wt % free alcohols (from the hydrolysis reaction). The water content of the product is preferably less than about 1% by weight, even more preferably less than about 0.1% by weight, and most preferably less than about 0.01% by weight.

The organic silicon compounds (a) are reactive compounds. In this context, it has been found preferable if the agent as contemplated herein contains—based on its total weight—one or more organic silicon compounds (a) in a total amount of about 0.1 to about 20.0% by weight, preferably from about 0.2 to about 15.0% by weight and particularly preferably about 0.2 to about 2.0% by weight. These quantities refer to the total weight of a ready-to-use mixture or—if used in the form of separate formulations—to the total weight of the respective formulation used for coloring the keratinous material. In general, all the quantitative data used herein have the meaning in the context of the composition according to the first aspect of the present disclosure, i.e., not the multicomponent kit.

It has proved particularly suitable to use in the composition as contemplated herein at least one organic silicon compound (a) obtainable by partial condensation of monomeric aminotrialkoxysilanes (a1) having one silane group, i.e., one silicon atom per molecule, and monomeric trialkoxysilanes (a2). For example, it may be preferred to use aminopropyltri(m)ethoxy silane as component (a1) in combination with an alkyltrialkoxysilane, for example alkyltri(m)ethoxy silane, especially C₁₋₃ alkyltri(m)ethoxy silane as component (a2), to obtain the organic silicon compound (a).

### Coloring Compounds

As a second ingredient (b) essential to the present disclosure, the compositions as contemplated herein contain at least one coloring compound. This is preferably selected from the group of photochromic dyes, thermochromic dyes, pigments and/or direct dyes, preferably from pigments and/or direct dyes.

Pigments within the meaning of the present disclosure are colorant compounds which have a solubility in water at about 25° C. of less than about 0.5 g/L, preferably less than about 0.1 g/L, still more preferably less than about 0.05 g/L. Water solubility can be determined, for example, by the method described below: about 0.5 g of the pigment are weighed in a beaker. A stir-fish is added. Then one liter of distilled water is added. This mixture is heated to about 25° C. for one hour with stirring on a magnetic stirrer. If undissolved components of the pigment are still visible in the mixture after this period, the solubility of the pigment is below about 0.5 g/L. If the pigment-water mixture cannot be assessed visually due to the high intensity of the pigment, which may be finely dispersed, the mixture is filtered. If a proportion of undissolved pigments remains on the filter paper, the solubility of the pigment is below about 0.5 g/L.

Suitable color pigments can be of inorganic and/or organic origin.

In a preferred embodiment, an agent as contemplated herein exemplified contains (b) at least one coloring compound from the group of inorganic and/or organic pigments.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulphides, complex metal cyanides, metal sulphates, chromates and/or molybdates. Particularly preferred color pigments are black iron oxide (CI 77499), yellow iron oxide (CI 77492), red and brown iron oxide (CI 77491), manganese violet (CI 77742), ultramarines (sodium aluminum sulfosilicates, CI 77007, Pigment Blue 29), chromium oxide hydrate (CI77289), iron blue (ferric ferrocyanide, CI77510) and/or carmine (cochineal).

Colored pearlescent pigments are also particularly preferred colorants from the group of pigments as contemplated herein. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred embodiment, an agent as contemplated herein exemplified comprises (b) at least one colorant compound from the group of pigments selected from the group of colored metal oxides, metal hydroxides, metal oxide hydrates, silicates, metal sulfides, complex metal cyanides, metal sulfates, bronze pigments and/or from mica- or mica-based colorant compounds coated with at least one metal oxide and/or a metal oxychloride.

In a further preferred embodiment, a composition as contemplated herein exemplified comprises (b) at least one colorant compound selected from mica- or mica-based pigments reacted with one or more metal oxides selected from the group of titanium dioxide (CI 77891), black iron oxide (CI 77499), yellow iron oxide (CI 77492), red and/or brown iron oxide (CI 77491, CI 77499), manganese violet (CI 77742), ultramarines (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly preferred color pigments with the trade name Colorona® are, for example:
Colorona Copper, Merck, MICA, CI 77491 (IRON OXIDES)
Colorona Passion Orange, Merck, Mica, CI 77491 (Iron Oxides), Alumina
Colorona Patina Silver, Merck, MICA, CI 77499 (IRON OXIDES), CI 77891 (TITANIUM DIOXIDE) Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES) Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE) Colorona Russet, Merck, CI 77491 (TITANIUM DIOXIDE), MICA, CI 77891 (IRON OXIDES) Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE Colorona Blackstar Green, Merck, MICA, CI 77499 (IRON OXIDES) Colorona Bordeaux, Merck, MICA, CI 77491 (IRON OXIDES)
Colorona Bronze, Merck, MICA, CI 77491 (IRON OXIDES) Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA Colorona Sienna, Merck, MICA, CI 77491 (IRON OXIDES)
Colorona Precious Gold, Merck, Mica, CI 77891 (Titanium dioxide), Silica, CI 77491 (Iron oxides), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491(Iron oxides)
Colorona Blackstar Gold, Merck, MICA, CI 77499 (IRON OXIDES)

Further particularly preferred color pigments with the trade name Xirona® are, for example: Xirona Golden Sky, Merck, Silica, CI 77891 (Titanium Dioxide), Tin Oxide Xirona Caribbean Blue, Merck, Mica, CI 77891 (Titanium Dioxide), Silica, Tin Oxide Xirona Kiwi Rose, Merck, Silica, CI 77891 (Titanium Dioxide), Tin Oxide Xirona Magic Mauve, Merck, Silica, CI 77891 (Titanium Dioxide), Tin Oxide.

In addition, particularly preferred color pigments with the trade name Unipure® are, for example: Unipure Red LC 381 EM, Sensient CI 77491 (Iron Oxides), Silica Unipure Black LC 989 EM, Sensient, CI 77499 (Iron Oxides), Silica Unipure Yellow LC 182 EM, Sensient, CI 77492 (Iron Oxides), Silica

In a further embodiment, the composition as contemplated herein may also contain (b) one or more coloring compounds from the group of organic pigments

The organic pigments of the present disclosure are correspondingly insoluble organic dyes or colorants which may be selected, for example, from the group of nitroso-, nitro-azo-, xanthene-, anthraquinone-, isoindolinone-, isoindoline-, quinacridone-, perinone-, perylene-, diketo-pyrrolopyorrole-, indigo-, thioindido-, dioxazine-, and/or triarylmethane compounds.

Particularly suitable organic pigments include, for example, carmine, quinacridone, phthalocyanine, sorghum, blue pigments with the color index numbers Cl 42090, CI 69800, CI 69825, CI 73000, CI 74100, CI 74160, yellow pigments with the color index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470 may be mentioned.

In a further particularly preferred embodiment, an agent as contemplated herein exemplified comprises (b) at least one color-imparting compound from the group of organic pigments selected from the group of carmine, quinacridone, phthalocyanine, sorghum, blue pigments having the Color Index numbers Cl 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments having the Color.

Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color paint. As contemplated herein, the term color lacquer means particles comprising a layer of absorbed dyes, the unit of particle and dye being insoluble under the above-mentioned conditions. The particles can, for example, be inorganic substrates, which can be aluminum, silica, calcium borosilate, calcium aluminum borosilicate or even aluminum.

For example, alizarin color varnish can be used.

Due to their excellent resistance to light and temperature, the use of the pigments in the composition as contemplated herein is particularly preferred. It is also preferred if the pigments used have a certain particle size. This particle size leads on the one hand to an even distribution of the pigments in the formed polymer film and on the other hand avoids a rough hair or skin feeling after application of the cosmetic product. As contemplated herein, it is therefore advantageous if the at least one pigment has an average particle size D50 of about 1.0 to about 50 μm, preferably from about 5.0 to about 45 μm, preferably from about 10 to about 40 μm, about 14 to about 30 μm. The mean particle size D50 can be determined using dynamic light scattering (DLS) as an example.

The pigment or pigments (b) may be used in an amount of from about 0.001 to about 20% by weight, from about 0.05 to about 5% by weight, in each case based on the total weight of the inventive agent.

As colorant compounds (b), the compositions as contemplated herein may also contain one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at about 25° C. of more than about 0.5 g/L and are therefore not to be regarded as pigments. Preferably, the direct dyes within the meaning of the present disclosure have a solubility in water (760 mmHg) at about 25° C. of more than about 1.0 g/L. In particular, the direct dyes within the meaning of the present disclosure have a solubility in water (760 mmHg) at about 25° C. of more than about 1.5 g/L.

Direct dyes can be divided into anionic, cationic, and nonionic direct dyes.

In a further preferred embodiment, an agent as contemplated herein exemplified contains as coloring compound (b) at least one anionic, cationic and/or non-ionic direct dye.

In a further preferred embodiment, an agent as contemplated herein exemplified contains (b) at least one anionic, cationic and/or non-ionic direct dye.

Suitable cationic direct dyes include Basic Blue 7, Basic Blue 26, Basic Violet 2, and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76

As non-ionic direct dyes, non-ionic nitro and quinone dyes and neutral azo dyes can be used. Suitable nonionic direct-acting dyes are those known under the international designations or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)amino-2-nitrobenzene, 3-nitro-4-(2-hydroxyethyl)-aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]-benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitro-benzoic acid and 2-chloro-6-ethylamino-4-nitrophenol.

In various embodiments, colorations with particularly high color intensity can be produced with agents comprising (b) at least one anionic direct dye.

In an explicitly particularly preferred embodiment, an agent as contemplated herein exemplified contains (b) at least one anionic direct dye.

Anionic direct dyes are also called acid dyes. Acid dyes are direct dyes which have at least one carboxylic acid grouping (—COOH) and/or one sulfonic acid grouping (—SO₃H). Depending on the pH, the protonated forms (—COOH, —SO₃H) of the carboxylic acid or sulfonic acid groupings are in equilibrium with their deprotonated forms (—COO—, —SO— present). The proportion of protonated forms increases with decreasing pH. If direct dyes are used in the form of their salts, the carboxylic acid groups or sulphonic acid groups are present in deprotonated form and are neutralized with corresponding stoichiometric equivalents of cations to maintain electro neutrality. Inventive acid dyes can also be used in the form of their sodium salts and/or their potassium salts.

The acid dyes within the meaning of the present disclosure have a solubility in water (760 mmHg) at about 25° C. of more than about 0.5 g/L and are therefore not to be regarded as pigments. Preferably the acid dyes within the meaning of the present disclosure have a solubility in water (760 mmHg) at about 25° C. of more than about 1.0 g/L.

The alkaline earth salts (such as calcium salts and magnesium salts) or aluminum salts of acid dyes often have a lower solubility than the corresponding alkali salts. If the solubility of these salts is below about 0.5 g/L (25° C., 760 mmHg), they do not fall under the definition of a direct dye.

An essential characteristic of acid dyes is their ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

In the context of an embodiment, an agent for coloring keratinous material is thus preferred, which exemplified(b) at least one anionic direct dye selected from the group of nitrophenylenediamines, nitroaminophenols, azo dyes, anthraquinone dyes, triarylmethane dyes, xanthene dyes, rhodamine dyes, oxazine dyes and/or indophenol dyes, the dyes from the abovementioned group each having at least one carboxylic acid group (—COOH), a sodium carboxylate group (—COONa), a potassium carboxylate group (—COOK), a sulfonic acid group (—SO₃H), a sodium sulfonate group (—SO₃Na) and/or a potassium sulfonate group (—SO₃K).

For example, one or more compounds from the following group can be selected as particularly well suited acid dyes: Acid Yellow 1 (D&C Yellow 7, Citronin A, Ext. D&C Yellow No. 7, Japan Yellow 403, CI 10316, COLIPA n° B001), Acid Yellow 3 (COLIPA n°: C 54, D&C Yellow N° 10, Quinoline Yellow, E104, Food Yellow 13), Acid Yellow 9 (CI 13015), Acid Yellow 17 (CI 18965), Acid Yellow 23 (COLIPA n° C. 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA n° C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; no sodium salt; Brown No. 201; RESORCIN BROWN; ACID ORANGE 24; Japan Brown 201; D & C Brown No. 1), Acid Red 14 (C.I. 14720), Acid Red 18 (E124, Red 18; CI 16255), Acid Red 27 (E 123, CI 16185, C-Rot 46, Echtrot D, FD&C Red Nr. 2, Food Red 9, Naphtholrot S), Acid Red 33 (Red 33, Fuchsia Red, D&C Red 33, CI 17200), Acid Red 35 (CI C.I. 18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red n° 106 Pontacyl Brilliant Pink), Acid Red 73 (CI 27290), Acid Red 87 (Eosin, CI 45380), Acid Red 92 (COLIPA n° C53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet n° 2, C.I. 60730, COLIPA n° C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent blue AE, Amido blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Food green 1), Acid Green 5 (CI 42095), Acid Green 9 (C.I. 42100), Acid Green 22 (C.I. 42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black n° 401, Naphthalene Black 10B, Amido Black 10B, CI 20 470, COLIPA n° B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. about 0.1 g of the anionic direct dye is placed in a beaker. A stir-fish is added. Then add about 100 ml of water. This mixture is heated to about 25° C. on a magnetic stirrer while stirring. It is stirred for about 60 minutes. The aqueous mixture is then visually assessed. Are there any unresolved residues, the amount of water is increased—for example in steps of about 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If about 0.1 g of the anionic direct dye dissolves in about 100 ml water at about 25° C., the solubility of the dye is about 1.0 g/L.

Acid Yellow 1 is called 8-hydroxy-5,7-dinitro-2-naphthalenesulfonic acid disodium salt and has a solubility in water of at least 40 g/L (25° C.).
Acid Yellow 3 is a mixture of the sodium salts of monosulfonic and sisulfonic acids of 2-(2-quinolyl)-1H-indene-1,3(2H)-dione and has a water solubility of 20 g/L (25° C.).
Acid Yellow 9 is the disodium salt of 8-hydroxy-5,7-dinitro-2-naphthalenesulfonic acid, its solubility in water is above 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid and is readily soluble in water at 25° C.
Acid Orange 7 is the sodium salt of 4-[(2-hydroxy-1-naphthyl)azo]benzene sulphonate. Its water solubility is more than 7 g/L (25° C.).
Acid Red 18 is the trinatirum salt of 7-hydroxy-8-[(E)-(4-sulfonato-1-naphthyl)-diazenyl)]-1,3-naphthalene disulfonate and has a very high-water solubility of more than 20% by weight. Acid Red 33 is the diantrium salt of 5-amino-4-hydroxy-3-(phenylazo)-naphthalene-2,7-disulphonate, its solubility in water is 2.5 g/L (25° C.).
Acid Red 92 is the disodium salt of 3,4,5,6-tetrachloro-2-(1,4,5,8-tetrabromo-6-hydroxy-3-oxoxanthen-9-yl)benzoic acid, whose solubility in water is reported to be greater than 10 g/L (25° C.).
Acid Blue 9 is the disodium salt of 2-({4-[N-ethyl(3-sulfonatobenzyl]amino]phenyl}{4-[(N-ethyl(3-sulfonatobenzyl)imino]-2,5-cyclohexadien-1-ylidene}methyl)-benzenesulfonate and has a water solubility greater than 20% by weight (25° C.).

A very particularly preferred composition as contemplated herein exemplified comprises (b) at least one anionic direct dye selected from the group of Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

The direct dyestuff(s), in particular the anionic direct dyestuff(s), can be used in different quantities in the means according to the desired color intensity. Particularly good results were obtained when the agent as contemplated herein contains—based on its total weight—one or more direct dyes (b) in a total amount of 0.01 to 10.0% by weight, preferably from about 0.1 to about 8.0% by weight, more preferably from about 0.2 to about 6.0% by weight and very particularly preferably from about 0.5 to about 4.5% by weight.

In a further preferred embodiment, an agent as contemplated herein exemplifiedcontains—based on its total weight—one or more direct dyes (b) in a total amount of from about 0.01 to about 10.0% by weight, preferably from about 0.1 to about 8.0% by weight, more preferably from about 0.2 to about 6.0% by weight and very particularly preferably from about 0.5 to about 4.5% by weight.

In a further preferred embodiment, an agent as contemplated herein exemplifiedcontains—based on its total weight—one or more anionic direct dyes (b) in a total amount of from about 0.01 to about 10.0% by weight, preferably from about 0.1 to about 8.0% by weight, more preferably from about 0.2 to about 6.0% by weight and very preferably from about 0.5 to about 4.5% by weight.

Thermochromic dyes can also be used. Thermochromism involves the property of a material to change its color reversibly or irreversibly as a function of temperature. This can be done by changing both the intensity and/or the wavelength maximum.

Finally, it is also possible to use photochromic dyes. Photochromism involves the property of a material to change its color depending reversibly or irreversibly on irradiation with light, especially UV light. This can be done by changing both the intensity and/or the wavelength maximum.

### Film-Forming Polymer

The compositions as contemplated herein may further comprise at least one film-forming polymer as a third ingredient (c). This polymer may be present in a further separate formulation, spatially separated from the formulations of ingredients (a) and (b), or pre-formulated together with the colorant compound (b).

Polymers are macromolecules with a molecular weight of at least about 1000 g/mol, preferably of at least about 2500 g/mol, particularly preferably of at least about 5000 g/mol, which include identical, repeating organic units. The polymers of the present disclosure may be synthetically produced polymers which are manufactured by polymerization of one type of monomer or by polymerization of different types of monomer which are structurally different from each other. If the polymer is produced by polymerization of a monomer type, they are referred to as homopolymers. If structurally different monomer types are used in polymerization, the resulting polymer is called a copolymer.

The maximum molecular weight of the polymer depends on the degree of polymerization (number of polymerized monomers) and the batch size and is determined by the polymerization method. In terms of the present disclosure, it is preferred if the maximum molecular weight of the film-forming hydrophobic polymer (c) is not more than about 10⁷ g/mol, preferably not more than about 10⁶ g/mol and particularly preferably not more than about 10⁵ g/mol.

A hydrophilic polymer is defined as a polymer that has a solubility in water at about 25° C. (760 mmHg) of more than about 1% by weight, preferably more than about 2% by weight. A hydrophobic polymer is a polymer that has a solubility in water at about 25° C. (760 mmHg) of less than about 1% by weight.

The water solubility of the film-forming polymer can be determined, for example, in the following way. about 1.0 g of the polymer is placed in a beaker. Make up to about 100 g with water. A stir-fish is added, and the mixture is heated to about 25° C. on a magnetic stirrer while stirring. It is stirred for about 60 minutes. The aqueous mixture is then visually assessed. A completely dissolved polymer appears macroscopically homogeneous. If the polymer-water mixture cannot be assessed visually due to a high turbidity of the mixture, the mixture is filtered. If no undissolved polymer remains on the filter paper, then the solubility of the polymer is more than about 1 wt %; if undissolved polymer remains, then the solubility of the polymer is less than about 1 wt %.

As contemplated herein, a film-forming polymer is a polymer which can form a film on a substrate, for example on a keratinic material or a keratinic fiber. The formation of a film can be demonstrated, for example, by viewing the polymer-treated keratin material under a microscope.

Nonionic, anionic, and cationic polymers can be used as film-forming polymers.

These include acrylic acid-type polymers, polyurethanes, polyesters, polyamides, polyureas, cellulose polymers, nitrocellulose polymers, silicone polymers, acrylamide-type polymers, and polyisoprenes.

Well-suited film-forming, hydrophobic polymers are, for example, polymers from the group of copolymers of acrylic acid, copolymers of methacrylic acid, homopolymers or copolymers of acrylic acid esters, homopolymers or copolymers of methacrylic acid esters, the homopolymers or copolymers of acrylic acid amides, of homopolymers or copolymers of methacrylic acid amides, of copolymers of vinylpyrrolidone, of copolymers of vinyl alcohol, of copolymers of vinyl acetate, homopolymers or copolymers of ethylene, homopolymers or copolymers of propylene, homopolymers or copolymers of styrene, polyurethanes, polyesters and/or polyamides.

Suitable film-forming hydrophilic polymers can be selected, for example, from the group of polyvinyl pyrrolidone (co)polymers, polyvinyl alcohol (co)polymers, vinyl acetate (co)polymers, carboxyvinyl (co)polymers, acrylic acid (co)polymers, methacrylic acid (co)polymers, natural gums, polysaccharides and/or acrylamide (co)polymers.

For example, polyvinylpyrrolidone (PVP) and/or a copolymer containing vinylpyrrolidone can be used as the film-forming hydrophilic polymer.

In various embodiments, an agent as contemplated herein comprises at least one film-forming hydrophilic polymer selected from the group of polyvinylpyrrolidone (PVP) and the copolymers of polyvinylpyrrolidone.

Polyvinylpyrrolidone as a film-forming, hydrophilic polymer (c) is extremely easy and simple to dissolve in water and keeps larger application quantities of pigments stable in dispersion over a long time. The wash fastness of the dyeing's that can be obtained with PVP-containing formulations is also particularly good.

Particularly well-suited polyvinylpyrrolidones are available, for example, under the name Luviskol® K from BASF SE, especially Luviskol® K 90 or Luviskol® K 85 from BASF SE.

The polymer PVP K30, which is marketed by Ashland (ISP, POI Chemical), can also be used as another explicitly very well suited polyvinylpyrrolidone (PVP). PVP K 30 is a polyvinylpyrrolidone that is very soluble in cold water and has the CAS number 9003-39-8. The molecular weight of PVP K 30 is about 40000 g/mol.

Other particularly well-suited polyvinylpyrrolidones are those sold under the trade names LUVITEC K 17, LUVITEC K 30, LUVITEC K 60, LUVITEC K 80, LUVITEC K 85, LUVITEC K 90 and LUVITEC K 115 substances known and available from BASF.

It is also possible to use film-forming hydrophilic polymers (c) from the group of copolymers of polyvinylpyrrolidone, which also lead to good and washfast color results. The storage stability of the formulations containing one or more copolymers of polyvinylpyrrolidone (c) is also particularly good.

In this context, vinylpyrrolidone-vinyl ester copolymers, such as those marketed under the trademark Luviskol® (BASF), can be mentioned as particularly suitable film-forming, hydrophilic polymers. Luviskol® VA 64 and Luviskol® VA 73, both vinylpyrrolidone/vinyl acetate copolymers, are particularly preferred non-ionic polymers.

Of the vinylpyrrolidone-containing copolymers, a styrene/VP copolymer and/or a vinylpyrrolidone-vinyl acetate copolymer and/or a VP/DMAPA acrylates copolymer and/or a VP/vinyl caprolactam/DMAPA acrylates copolymer are particularly preferred in cosmetic compositions.

Vinylpyrrolidone-vinyl acetate copolymers are marketed under the name Luviskol® VA by BASF SE. For example, a VP/Vinyl Caprolactam/DMAPA Acrylates copolymer is sold under the trade name Aquaflex® SF-40 by Ashland Inc. For example, a VP/DMAPA acrylates copolymer is marketed by Ashland under the name Styleze CC-10 and is a highly preferred vinylpyrrolidone-containing copolymer.

Other suitable copolymers of polyvinylpyrrolidone (c) may include those obtained by reacting N-vinylpyrrolidone with at least one further monomer selected from the group of V-vinylformamide, vinyl acetate, ethylene, propylene, acrylamide, vinylcaprolactam, vinyl-caprolactone and/or vinyl alcohol.

In various embodiments, an agent (c) as contemplated herein comprises at least one film-forming hydrophilic polymer selected from the group of polyvinylpyrrolidone (PVP), vinylpyrrolidone/vinyl acetate copolymers, vinylpyrrolidone/styrene copolymers, vinylpyrrolidone/ethylene copoylmeres, vinylpyrrolidone/propylene copolymers, vinylpyrrolidone/vinylcaprolactam copolymers, vinylpyrrolidone/vinylformamide copolymers and/or vinylpyrrolidone/vinyl alcohol copolymers.

Another fussy copolymer of vinylpyrrolidone is the polymer known under the INCI designation maltodextrin/VP copolymer.

Furthermore, intensively colored keratin material, especially hair, with particularly good wash fastness properties can be obtained if a nonionic, film-forming, hydrophilic polymer is used as the film-forming, hydrophilic polymer.

In various embodiments, an agent as contemplated herein exemplified comprises (c) at least one nonionic, film-forming, hydrophilic polymer.

As contemplated herein, a nonionic polymer is a polymer which, in a protic solvent—such as water—does not, under standard conditions, carry structural units with permanent cationic or anionic groups which can be removed by counterions. must be compensated while maintaining electroneutrality. Cationic groups include quaternized ammonium groups but not protonated amines Anionic groups include carboxylic and sulphonic acid groups.

In various embodiments, the compositions comprise, as a nonionic, film-forming, hydrophilic polymer, at least one polymer selected from the group of.

Polyvinylpyrrolidone,

Copolymers of N-vinylpyrrolidone and vinyl esters of carboxylic acids containing about 2 to about 18 carbon atoms of N-vinylpyrrolidone and vinyl acetate,

Copolymers of N-vinylpyrrolidone and N-vinylimidazole and methacrylamide,

Copolymers of N-vinylpyrrolidone and N-vinylimidazole and acrylamide,

Copolymers of N-vinylpyrrolidone with N,N-di(C1 to C4)-alkylamino-(C2 to C4)-alkyl acrylamide.

If copolymers of N-vinylpyrrolidone and vinyl acetate are used, it is again preferable if the molar ratio of the structural units contained in the monomer N-vinylpyrrolidone to the structural units of the polymer contained in the monomer vinyl acetate is in the range from about 20:80 to about 80:20, in particular from about 30:70 to about 60:40. Suitable copolymers of vinyl pyrrolidone and vinyl acetate are available, for example, under the trademarks Luviskol® VA 37, Luviskol® VA 55, Luviskol® VA 64 and Luviskol® VA 73 from BASF SE.

Another particularly preferred polymer is selected from polymers with the INCI designation VP/Methacrylamide/Vinyl Imidazole Copolymer, which are available, for example, under the trade name Luviset Clear from BASF SE.

Another preferred nonionic, film-forming, hydrophilic polymer is a copolymer of N-vinylpyrrolidone and N,N-dimethylaminopropylmethacrylamide, which is sold, for example, by ISP under the INCI name VP/DMAPA Acrylates Copolymer, e.g., under the trade name Styleze® CC 10.

A cationic polymer useful as contemplated herein is the copolymer of N-vinylpyrrolidone, N-vinylcaprolactam, N-(3-dimethylaminopropyl)methacrylamide and 3-(methacryloylamino)propyl-lauryl-dimethylammonium chloride (INCI name: Polyquaternium-69), which is marketed, for example, under the trade name AquaStyle® 300 (28-32 wt. % active substance in ethanol-water mixture, molecular weight 350000) by ISP.

Other suitable film-forming, hydrophilic polymers include

Vinylpyrrolidone-vinylimidazolium methochloride copolymers, as offered under the designations Luviquat^{□} FC 370, FC 550 and the INCI designation Polyquaternium-16 as well as FC 905 and HM 552,

Vinylpyrrolidone-vinylcaprolactam-acrylate terpolymers, as they are commercially available with acrylic acid esters and acrylic acid amides as a third monomer component, for example under the name Aquaflex® SF 40.

Polyquaternium-11 is the reaction product of diethyl sulphate with a copolymer of vinyl pyrrolidone and dimethylaminoethyl methacrylate. Suitable commercial products are available under the names Dehyquart® CC 11 and Luviquat® PQ 11 PN from BASF SE or Gafquat 440, Gafquat 734, Gafquat 755 or Gafquat 755N from Ashland Inc.

Polyquaternium-46 is the reaction product of vinylcaprolactam and vinylpyrrolidone with methylvinylimidazolium methosulfate and is available for example under the name Luviquat® Hold from BASF SE. Polyquaternium-46 is preferably used in an amount of 1 to 5% by weight—based on the total weight of the cosmetic composition. It particularly prefers to use polyquaternium-46 in combination with a cationic guar compound. It is even highly preferred that polyquaternium-46 is used in combination with a cationic guar compound and polyquaternium-11.

Suitable anionic film-forming polymers can be, for example, acrylic acid polymers, which can be in uncrosslinked or crosslinked form. Such products are sold commercially under the trade names Carbopol 980, 981, 954, 2984 and 5984 by Lubrizol or under the names Synthalen M and Synthalen K by 3V Sigma (The Sun Chemicals, Inter Harz).

Examples of suitable film-forming, hydrophilic polymers from the group of natural gums are xanthan gum, gellan gum, carob gum.

Examples of suitable film-forming hydrophilic polymers from the group of polysaccharides are hydroxyethyl cellulose, hydroxypropyl cellulose, ethyl cellulose and carboxymethyl cellulose.

Suitable film-forming, hydrophilic polymers from the acrylamide group are, for example, polymers prepared from monomers of (methy)acrylamido-C1-C4-alkyl-sulfonic acid or salts thereof. Corresponding polymers may be selected from the polymers of polyacrylamidomethanesulfonic acid, polyacrylamidoethanesulfonic acid, polyacrylamido-propanesulfonic acid, poly2-acrylamido-2-methylpropanesulfonic acid, poly-2-methylacrylamido-2-methylpropanesulfonic acid and/or poly-2-methylacrylamido-n-butanesulfonic acid.

Preferred polymers of poly(meth)arylamido-C1-C4-alkyl-sulfonic acids are crosslinked and at least about 90% neutralized. These polymers can be crosslinked or non-crosslinked.

Cross-linked and fully or partially neutralized polymers of the poly-2-acrylamido-2-methylpropane sulfonic acid type are known under the INCI names “Ammonium Polyacrylamido-2-methyl-propane-esulphonate ” or “Ammonium Polyacryldimethyltauramide”.

Another preferred polymer of this type is the cross-linked poly-2-acrylamido-2-methyl-propanesulphonic acid polymer marketed by Clamant under the trade name Hostacerin AMPS, which is partially neutralized with ammonia.

In a further preferred embodiment, an agent as contemplated herein comprises at least one film-forming, hydrophobic polymer (c) which is selected from the group of the copolymers of acrylic acid, the copolymers of methacrylic acid, the homopolymers or copolymers of acrylic acid esters, the homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

Other particularly well-suited film-forming hydrophobic polymers can be selected from the homopolymers or copolymers of olefins, such as cycloolefins, butadiene, isoprene or styrene, vinyl ethers, vinylamides, the esters or amides of (meth)acrylic acid with at least one C₁-C₂₀ alkyl group, an aryl group or a C2-C10 hydroxyalkyl group.

Other film-forming hydrophobic polymers may be selected from the homo- or copolymers of isooctyl (meth)acrylate; isononyl (meth)acrylate; 2-ethylhexyl(meth)acrylate; lauryl (meth)acrylate); isopentyl (meth)acrylate; n-butyl (meth)acrylate); isobutyl (meth)acrylate; ethyl (meth)acrylate; methyl (meth)acrylate; tert-butyl (meth)acrylate; stearyl (meth)acrylate; hydroxyethyl (meth)acrylate; 2-hydroxypropyl (meth)acrylate; 3-hydroxypropyl (meth)acrylate; and/or mixtures thereof.

Other film-forming hydrophobic polymers may be selected from the homo- or copolymers of (meth)acrylamide; N-alkyl-(meth)acrylamides, in those with C2-C18 alkyl groups, such as N-ethyl-acrylamide, N-tert-butyl-acrylamide, le N-octyl-crylamide; N-di(C1-C4)alkyl-(meth)acrylamide.

Preferred anionic copolymers are, for example, copolymers of acrylic acid, methacrylic acid or their C₁-C₆ alkyl esters, as they are marketed under the INCI Declaration Acrylates Copolymers. A suitable commercial product is for example Aculyn® 33 from Rohm & Haas. Copolymers of acrylic acid, methacrylic acid or their C₁-C₆ alkyl esters and the esters of an ethylenically unsaturated acid and an alkoxylated fatty alcohol are also preferred. Suitable ethylenically unsaturated acids are especially acrylic acid, methacrylic acid and itaconic acid; suitable alkoxylated fatty alcohols are especially steareth-20 or ceteth-20.

Very particularly preferred polymers on the market are, for example, Aculyn® 22 (Acrylates/Steareth-20 Me-thacrylate Copolymer), Aculyn® 28 (Acrylates/Beheneth-25 Methacrylate Copolymer), Structure 2001® (Acryla-tes/Steareth-20 Itaconate Copolymer), Structure 3001® (Acrylates/Ceteth-20 Itaconate Copolymer), Structure Plus® (Acrylates/Aminoacrylates C10-30 Alkyl PEG-20 Itaconate Copolymer), Carbopol® 1342, 1382, Ultrez 20, Ultrez 21 (Acrylates/C10-30 Alkyl Acrylate Crosspolymer), Synthalen W 2000® (Acrylates/Palmeth-25 Acrylate Copolymer) or the Rohme and Haas distributed Soltex OPT (Acrylates/C12-22 Alkyl methacrylate Copolymer).

The homo- and copolymers of N-vinylpyrrolidone, vinylcaprolactam, vinyl-(C1-C6)alkyl-pyrrole, vinyl-oxazole, vinyl-thiazole, vinylpyrimidine, vinylimidazole can be named as suitable polymers based on vinyl monomers.

Also particularly suitable are the copolymers octylacrylamide/acrylates/butylamino-ethyl methacrylate copolymer, such as those sold commercially under the trade names AMPHOMER® or LOVOCRYL® 47 from NATIONAL STARCH, or the copolymers of acrylates/octylacrylamides sold under the trade names DERMACRYL® LT and DERMACRYL® 79 from NATIONAL STARCH.

Suitable olefin-based polymers include homopolymers and copolymers of ethylene, propylene, butene, isoprene and butadiene.

In another embodiment, the film-forming hydrophobic polymers may be the block copolymers comprising at least one block of styrene or the derivatives of styrene. These block copolymers can be copolymers that contain one or more other blocks in addition to a styrene block, such as styrene/ethylene, styrene/ethylene/butylene, styrene/butylene, styrene/isoprene, styrene/butadiene. Such polymers are commercially distributed by BASF under the trade name “Luvitol HSB”.

In various embodiments, particularly good colorations can be obtained with the anionic direct dyes when the film-forming hydrophobic polymer also carries anionic charges.

In various embodiments, an agent as contemplated herein exemplified comprises (c) at least one anionic, film-forming, hydrophobic polymer.

An anionic polymer is a polymer comprising repeating units having at least one carboxylic acid group, one sulphonic acid group and/or their physiologically acceptable salts. In other words, an anionic polymer is made from monomers having at least one carboxylic acid group, a sulphonic acid group. In this context, the hydrophobic, film-forming (co)polymers of acrylic acid and the (co)polymers of methacrylic acid are particularly preferred. In the polymers of these groups are the carboxylic acid groups containing sulfonic acid groups or their salts in an amount that ensures that the hydrophobic character of the entire polymer is maintained.

In preferred embodiments, the film-forming polymer is a polymer or copolymer based on acrylic acid and/or methacrylic acid.

The film-forming polymer or polymers (c) are preferably used in certain ranges of amounts in the composition as contemplated herein. In this context, it has proved particularly preferable for solving the problem as contemplated herein if the agent contains—based on its total weight—one or more polymers in a total amount of about 0.1 to about 25.0 wt. %. %, preferably from about 0.2 to about 20.0 wt. %, further preferably from about 0.5 to about 15.0 wt. % and most preferably from about 1.0 to about 7.0 wt. %.

In a further preferred embodiment, an agent as contemplated herein exemplifiedcontains—based on its total weight—one or more film-forming polymers (c) in a total amount of from about 0.1 to about 25.0% by weight, preferably from about 0.2 to about 20.0% by weight, more preferably from about 0.5 to about 15.0% by weight and very particularly preferably from about 1.0 to about 7.0% by weight.

### Silicone

The compositions as contemplated herein may further comprise, in addition, at least one silicone as a fourth ingredient (d). This silicone, if present, may be pre-formulated together with the organic silicon compound (a) or the colorant compound (b).

The silicones optionally used to formulate the organic silicone compounds (a) are preferably volatile silicones, in particular siloxanes of the formula (IV) where

(R₁₀)₃Si—O—(Si(R₁₀)₂—O)ₖ—Si(R₁₀)₃ (IV),

each R₁₀ independently represents a C₁-C₆ alkyl group, preferably methyl or ethyl, more preferably methyl,

k is 0 or an integer from about 1 to about 30, preferably about 0 to about 10, more preferably 0-5, most preferably 0 or 1, i.e., hexamethyldisiloxane and octamethyltrisiloxane.

“Volatile silicones,” as used herein, means silicones with kinematic viscosities at 25° C. in the range 0.65-20.0 cSt (0.0065-0.2 cm²/s), more preferably silicones with 0.65-2.0 cSt, even more preferably up to 1.0 cSt. These have the property to evaporate quickly during application and do not influence the condensation reaction of the silanes. Such silicones are the methylsiloxanes of the above formula (IV) in which k=0 to 30 or 0 to 20 or 0 to 10, preferably from about 0 to about 3, more preferably from about 0 to about 1.

The silicones optionally used to formulate the coloring compounds (b) are preferably PEG-modified dimethylsiloxanes of formula (V)

(R₁₀)₃Si—O—(Si(R₁₀R₁₁)—O)ₖ—Si(R₁₀)₃ (V),

where

each R₁₀ independently represents a C₁-C₆ alkyl group, preferably methyl or ethyl, more preferably methyl,

each R₁₁ is independently a C₁-C₆ alkyl group, preferably methyl or ethyl, more preferably methyl, or a group of the formula —(CH₂)ₗ—(OCH₂CH₂)ₘOH,

k is an integer from about 1 to about 100, preferably from about 1 to about 30;

l is an integer from about 1 to about 10, preferably from about 2 or about 3;

m is an integer from about 1 to about 30, preferably from about 5 to about 20, more preferably from about 10, about 11, about 12, about 13 or about 14; wherein at least one R₁₁ but preferably not all R₁₁ is/are a group of the formula —(CH₂)ₗ—(OCH₂CH₂)ₘOH.

### Water Content of the Agents

The composition as contemplated herein contains the essential ingredients (a), (b) and optionally (c) in a cosmetic carrier, preferably in an aqueous or water-containing cosmetic carrier, as already described above.

Such products, in which the above ingredients are present in an aqueous or water-containing cosmetic carrier, are typically the ready-to-use products. Indeed, without being bound by this theory, the organic silicon compound (a) comprising one or more hydroxyl groups or hydrolysable groups per molecule is believed to further hydrolyze and/or condense in the presence of the water. The hydrolysis products or oligo- and/or polymers formed in this way have a particularly high affinity for the surface of the keratin material. With the colorant compounds (b), a stable and resistant film can optionally be formed in this way together with the film-forming hydrophilic polymer (c).

It is preferred as contemplated herein that ingredients (a) and (b) are formulated/mixed in an aqueous or water-containing carrier for use, and optional ingredient (c) is formulated separately in an aqueous or water-containing carrier for use and used in a subsequent step. Alternatively, it may also be preferred as contemplated herein to formulate ingredient (a) in an aqueous or water-containing carrier for use and to formulate/mix ingredient (b) and optionally also ingredient (c) separately in an aqueous or water-containing carrier for use and to use them in a subsequent step.

In various embodiments, therefore, the composition may have a water content—based on its total weight—of from about 15 to about 95% by weight, preferably from about 20 to about 95% by weight, more preferably from about 25 to about 95% by weight, still more preferably from about 30 to about 95% by weight, and most preferably from about 45 to about 95% by weight.

In a further embodiment, a composition as contemplated herein exemplified comprises.—based on its total weight—has a water content of from about 15 to about 95% by weight, preferably from about 20 to about 95% by weight, more preferably from about 25 to about 95% by weight, still more preferably from about 30 to about 95% by weight and very particularly preferably from about 45 to about 95% by weight.

### Multi-Component Packaging Unit (Kit-of-Parts)

The previously described agent of the first subject present disclosure may be the ready-to-use colorant. This contains with the organic silicon compound(s) (a) a class of reactive compounds which can undergo further hydrolysis and/or condensation in the presence of water as previously described.

To increase storage stability and to avoid premature complete hydrolysis and condensation, this agent is preferably provided to the user in the form of a multi-component packaging unit (kit-of-parts). Just before application to the keratinous material, the user can mix the various components of this packaging unit and in this way produce the ready-to-use colorant.

A second object of the present disclosure is a multi-component packaging unit (kit-of-parts) for dyeing keratinous material, in particular human hair, which separately includes

a first container containing a cosmetic product (I) and

a second container containing a cosmetic product (II) and

a third container containing a cosmetic product (III), wherein

the agent (I) comprises at least one organic silicon compound (a) as defined herein and optionally also a silicone as defined herein,

the composition (II) comprises water and optionally at least one colorant compound (b) as defined herein, and

the agent (III) comprises at least one film-forming polymer (c) as defined herein and optionally at least one color-imparting compound (b) as defined herein,

wherein either the agent (II), the agent (III) or both contain at least one color-imparting compound (b).

In embodiments in which agents (II) and (III) both comprise at least one colorant compound (b), the colorant compound (b) may be the same or different.

In various embodiments, the ready-to-use agent is prepared by mixing the agents (I) and (II) were prepared and agent (III) was applied separately after the application of agents (I) and (II). Alternatively, although not preferred, all three agents (I), (II), and (III) may be mixed to form the ready-to-use agent.

For example, the user can first mix or shake the agent (I) containing the organic silicon compound(s) (a) with the water-containing agent (II). This agent from (I) and (II) can then already be applied directly as such to the keratin-containing material, with agent (III) being applied in a subsequent step. Alternatively, after mixing agents (I) and (II), the user can optionally add agent (III), which may contain the colorant compound(s), to the mixture (b) and the film-forming polymer(s) (c) to the mixture of (I) and (II) and mix all three agents together and apply the resulting mixture.

In particularly preferred embodiments, agents (I), (II) and (III) are applied successively to the keratinous material so that the agents interact with each other only on the keratinous material.

For example, as described above, the user may first mix or shake the agent (I) containing the organic silicon compound(s) (a) with the aqueous agent (II). The user can now apply this mixture of (I) and (II) to the keratin materials—either directly after their production or after a short reaction time of about 10 seconds to about 20 minutes. After that, the user can now apply the agent (III), which contains the film-forming polymer (c), to the keratin material. It may be preferred that the agent (II) contains coloring compounds (b). In such embodiments, agent (III) also contain color-imparting compounds, but in various embodiments do not contain such compounds (b).

To provide a formulation that is as stable as possible during storage, the agent (I) itself is preferably packaged with low or no water.

In a preferred embodiment, a multicomponent packaging unit (kit-of-parts) as contemplated herein is exemplified in that the agent (I)—based on the total weight of the agent (I)—contains a water content of less than about 1% by weight, even more preferably less than about 0.1% by weight and very particularly preferably less than about 0.01% by weight.

In a further preferred embodiment, a multicomponent packaging unit as contemplated herein is exemplified in that the agent (I) comprises—based on the total weight of the agent (I)—one or more organic silicon compounds (a) in a total amount of about 20 to about 100.0% by weight, preferably from about 25 to about 90% by weight, further preferably from about 30 to about 80% by weight and most preferably from about 40 to about 75% by weight.

Agent (II) contains water. A multi-component packaging unit (kit-of-parts) as contemplated herein is exemplified in that the agent (II)—based on the total weight of the agent (II)—has a water content of about 15 to about 100% by weight, preferably of about 35 to about 100% by weight, more preferably of about 55 to about 100% by weight, still more preferably of about 65 to about 100% by weight and very particularly preferably of about 75 to about 100% by weight.

The agent (III) comprises at least one film-forming polymer (c) as already disclosed in detail in the description of the first subject matter of the present disclosure.

Both agents (II) and agents (III) may independently contain at least one colorant compound (b).

In a particularly preferred embodiment, the agent (II) and/or (III) contains the above-mentioned, the preferred and the particularly preferred pigments (b).

In a particularly preferred embodiment, the agent (II) and/or (III) contains the above-mentioned, the preferred and the particularly preferred direct dyes (b).

In a further preferred embodiment, a multicomponent packaging unit as contemplated herein is exemplified in that the agent (II) and/or (III) contains—based on the total weight of the agent (III)—one or more colorant compounds (b) in a total amount of from about 0.01 to about 10.0% by weight, preferably from about 0.1 to about 8.0% by weight, more preferably from about 0.2 to about 6.0% by weight and very particularly preferably from about 0.5 to about 4.5% by weight.

In a particularly preferred embodiment, the agent (III) contains the, the preferred and the particularly preferred film-forming polymers (c).

In a further preferred embodiment, a multicomponent packaging unit as contemplated herein is exemplified in that the agent (III) contains—based on the total weight of the agent (III)—one or more film-forming polymers (b) in a total amount of from about 0.1 to about 25.0% by weight, preferably from about 0.2 to about 20.0% by weight, more preferably from about 0.5 to about 15.0% by weight and very particularly preferably from about 1.0 to about 7.0% by weight.

Agents (I) and (II) or agents (I), (II) and (III) can be mixed in different quantities. For example, the first container may contain about 5 g to about 200 g of the agent (I). The second container can contain about 5 g to about 200 g of the agent (II). The third container can contain about 5 b to about 200 g of the agent (III).

### Other Ingredients

The previously described agent, i.e., the ready-to-use agent of the first present disclosure, and the agents (I), (II) and (III) of the kit as contemplated herein of the second present disclosure, may further contain one or more optional ingredients. These may comprise the silicones already described above in agents (I), (II) or (III), whereby for agent (I) the silicones preferred for formulation with the organic silicon compounds (a) are used here and for agents (II) or (III), depending on which contain the compounds (b), the silicones preferred for formulation with the color-imparting compounds (b).

The products may also contain one or more surfactants. The term surfactants refer to surface-active substances. A distinction is made between anionic surfactants of a hydrophobic residue and a negatively charged hydrophilic head group, amphoteric surfactants, which carry both a negative and a compensating positive charge, cationic surfactants, which in addition to a hydrophobic residue have a positively charged hydrophilic group, and non-ionic surfactants, which have no charges but strong dipole moments and are strongly hydrated in aqueous solution.

The term zwitterionic surfactants is used to describe surface-active compounds that carry at least one quaternary ammonium group and at least one —COO⁽⁻⁾ or —SO⁽⁻⁾ group in the molecule. Particularly suitable zwitterionic surfactants are the so-called betaines such as N-alkyl-N,N-dimethylammonium glycinates, for example cocoalkyl-dimethylammonium glycinate, N-acyl-aminopropyl-N,N-dimethylammonium glycinates, for example cocoacylaminopropyl-dimethylammonium glycinate, and 2-alkyl-3-carboxymethyl-3-hydroxyethyl-imidazolines, each having from about 8 to about 18 C atoms in the alkyl or acyl group and the cocosacylaminoethylhydroxyethylcarboxy-methylglycinate. A preferred zwitterionic surfactant is the fatty acid amide derivative known under the INCI name cocamidopropyl betaine.

Ampholytic surfactants are surface-active compounds which, in addition to a C₈-C₂₄ alkyl or acyl group in the molecule, contain at least one free amino group and at least one —COOH or —SO₃H group and can form internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with about 8 to 24 C atoms in the alkyl group. Typical examples of amphoteric or zwitterionic surfactants are alkyl betaines, alkylamidobetaines, amino-propionates, aminoglycinates, imidazo-linium betaines and sulfobetaines.

Particularly preferred ampholytic surfactants are N-cocosalkylaminopropionate, coco-acylaminoethylaminopropionate and C₁₂-C₁₈acyl sarcosine.

The products may also additionally contain at least one non-ionic surfactant. Suitable non-ionic surfactants are alkyl polyglycosides as well as alkylene oxide addition products to fatty alcohols and fatty acids with about 2 to about 30 mol ethylene oxide per mol fatty alcohol or fatty acid. Preparations with good properties are also obtained if they contain as non-ionic surfactants fatty acid esters of ethoxylated glycerol reacted with at least about 2 mol ethylene oxide. The nonionic surfactants are used in a total amount of about 0.1 to about 45% by weight, preferably from about 1 to about 30% by weight and very preferably from about 1 to about 15% by weight—based on the total weight of the respective agent—is used.

In addition, the products may also contain at least one cationic surfactant. Cationic surfactants are surfactants, i.e., surface-active compounds, each with one or more positive charges. Cationic surfactants contain only positive charges. Usually, these surfactants are composed of a hydrophobic part and a hydrophilic head group, the hydrophobic part usually including a hydrocarbon backbone (e.g., consisting of one or two linear or branched alkyl chains) and the positive charge(s) being in the hydrophilic head group. Examples of cationic surfactants are

quaternary ammonium compounds which may carry one or two alkyl chains with a chain length of about 8 to about 28 carbon atoms as hydrophobic radicals,

quaternary phosphonium salts substituted by one or more alkyl chains having a chain length of about 8 to about 28 carbon atoms or

tertiary sulfonium salts.

Furthermore, the cationic charge can also be part of a heterocyclic ring (e.g., an imidazolium ring or a pyridinium ring) in the form of an onium structure. In addition to the functional unit carrying the cationic charge, the cationic surfactant may also contain other uncharged functional groups, as is the case for example with esterquats. The cationic surfactants are used in a total amount of about 0.1 to about 45 wt. %, preferably from about 1 to about 30 wt. %. and very preferably from about 1 to about 15% by weight, based on the total weight of the respective agent.

Furthermore, the compositions as contemplated herein may also contain at least one anionic surfactant. Anionic surfactants are surface-active agents with exclusively anionic charges (neutralized by a corresponding counter cation). Examples of anionic surfactants are fatty acids, alkyl sulphates, alkyl ether sulphates and ether carboxylic acids with about 12 to about 20 C atoms in the alkyl group and up to about 16 glycol ether groups in the molecule.

The anionic surfactants are used in a total quantity of about 0.1 to about 45 wt. %, preferably from about 1 to about 30 wt. % and most preferably from about 1 to about 15 wt. %—based on the total weight of the respective agent.

The compositions may also contain other active ingredients, auxiliaries and additives, such as solvents, fatty ingredients such as C₈-C₃₀ fatty alcohols, C₈-C₃₀ fatty acid triglycerides, C₈-C₃₀ fatty acid monoglycerides, C₈-C₃₀ fatty acid diglycerides and/or hydrocarbons; structurants such as glucose, maleic acid and lactic acid, hair conditioning compounds such as phospholipids, for example lecitin and kephalins; perfume oils, dimethyl isosorbide and cyclodextrins; fiber structure-improving active ingredients, in particular mono-, di- and oligo-saccharides such as glucose, galactose, fructose, fructose and lactose; dyes for coloring the product; anti-dandruff active ingredients such as piroctone olamine, zinc omadine and climbazole; amino acids and oligopeptides; protein hydrolysates on an animal and/or vegetable basis, as well as in the form of their fatty acid condensation products or, optionally, anionically or cationically modified derivatives; vegetable oils; light stabilizers and UV blockers; active ingredients such as panthenol, pantothenic acid, pantolactone, allantoin, pyrrolidinone carboxylic acids and their salts, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; Vitamins, provitamins and vitamin precursors; plant extracts; fats and waxes such as fatty alcohols, beeswax, montan wax and kerosene's; swelling and penetrating agents such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono- and -distearate as well as PEG-3-distearate; and blowing agents such as propane-butane mixtures, N2O, dimethyl ether, CO2, and air.

The selection of these other substances will be made by the specialist according to the desired properties of the agents. About other optional components and the quantities of these components used, explicit reference is made to the relevant manuals known to the specialist. The additional active ingredients and auxiliary substances are preferably used in the preparations as contemplated herein in quantities of about 0.0001 to about 25 wt. % each, about 0.0005 to about 15 wt. %, based on the total weight of the respective agent.

### Process for Dyeing Keratin Materials

The agents described above—both the ready-to-use agents of the first present disclosure and the agents of the multi-component packaging unit of the second present disclosure as contemplated herein—are used in processes for dyeing keratinous materials, for dyeing human hair.

A third subject of the present disclosure is a process for dyeing keratinous material, in particular human hair, comprising the following steps in the order indicated:
(A)
(1) Application of a pretreatment agent (V) to the keratinous material, the pretreatment agent (V) comprising, in a water-containing cosmetic carrier, at least one organic silicon compound (a) and optionally also a silicone (d), as already disclosed in detail in the description of the first subject-matter of the present disclosure,
(2) Application of a coloring agent (F) to the keratinous material, the coloring agent comprising at least one coloring compound (b) and at least one film-forming polymer (c) as already disclosed in detail in the description of the first subject matter of the present disclosure. or, in another embodiment,
(B)
(1) Application of a pretreatment and coloring agent (VF) to the keratinous material, wherein the pretreatment and coloring agent (VF) contains in a water-containing cosmetic carrier at least one organic silicon compound (a), at least one coloring compound (b), and optionally also a silicone (d), as already disclosed in detail in the description of the first object of the present disclosure, and
(2) Application of an after-treatment agent (N) to the keratinous material, the after-treatment agent comprising at least one film-forming polymer (c), as already disclosed in detail in the description of the first subject-matter of the present disclosure, or, in another embodiment,
(C)
(1) Application of a pretreatment and coloring agent (VF) to the keratinous material, wherein the pretreatment and coloring agent (VF) contains in a water-containing cosmetic carrier at least one organic silicon compound (a), at least one coloring compound (b), and optionally also a silicone (d), as already disclosed in detail in the description of the first object of the present disclosure, and
(2) Application of a post-treatment and coloring agent (NF) to the keratinous material, the post-treatment and coloring agent comprising at least one coloring compound (b) and at least one film-forming polymer (c) as already disclosed in detail in the description of the first subject matter of the present disclosure.

In the process as contemplated herein, the keratin materials, in particular human hair, are first treated with a pretreatment agent (V) or preferably a pretreatment and coloring agent (VF). Subsequently, either the actual colorant (F) or a post-treatment agent (N) or a post-treatment and colorant (NF)

which contains the film-forming polymer and optionally the colorant compound(s)—is applied to the keratin materials.

Preferably, the pretreatment agent (V) itself contains the dyes or colorant compounds and is thus a pretreatment and colorant agent (VF). A characteristic feature of the pretreatment agent (V) and the pretreatment and coloring agent (VF) is its content of at least one reactive organic silicon compound (a). The reactive organic compound or compounds Silicon compounds (a) functionalize the hair surface as soon as they meet it. In this way, a first, colored or uncolored film is formed.

In the second step of the process, a coloring agent (F) or an after-treatment and coloring agent (NF) can now be applied to the hair. During the application of the colorant (F) or after-treatment and colorant (NF) to the keratin materials, a film is also formed on the—now already functionalized—hair surface, with (further) colorant compounds now being embedded in the film and thus deposited on the hair.

If a pretreatment and coloring agent (VF) has already been used in the first step, an aftertreatment agent (N), which does not contain coloring compounds, can also be applied to the hair in the second step of the process, resulting in further film formation on the—now already functionalized and colored—hair surface.

The film produced “in situ” in this way, in which the coloring compound is embedded, is exemplified by outstanding wash fastness and a homogeneous color result. The dyeing' s are glossy, and the feel of the dyed keratin materials is smooth and pleasant.

The pretreatment agent (V) or the pretreatment and coloring agent (VF) represents the pretreatment agent (V) or pretreatment and coloring agent (VF) ready for use. Very preferably, the pretreatment agent (V) or pretreatment and coloring agent (VF) is the mixture of agents (I) and (II) of the multi-component packaging unit as contemplated herein. The pretreatment agent (V) or pretreatment and coloring agent (VF) thus contains at least one organic silicon compound (a). Furthermore, the pretreatment agent (V) or pretreatment and coloring agent (VF) contains water, the water originating from the agent (II) of the kit-of-parts as contemplated herein.

In a very particularly preferred embodiment, a process as contemplated herein is exemplified in that the pretreatment agent (V) or pretreatment and coloring agent (VF) is prepared by mixing a first agent (I) and a second agent (II) prior to application to the keratinous material, wherein

the agent (I) comprises at least one organic silicon compound (a) as disclosed in detail in the description of the first and second present disclosures, and

the agent (II) comprises water and optionally at least one colorant compound as described herein.

It is preferred if the pretreatment agent (V) or pretreatment and coloring agent (VF)—based on the total weight of the pretreatment agent (V) or pretreatment and coloring agent (VF)—has a water content of from about 15 to about 95% by weight, preferably from about 20 to about 95% by weight, more preferably from about 25 to about 95% by weight, still more preferably from about 30 to about 95% by weight and very particularly preferably from about 45 to about 95% by weight.

In another very particularly preferred embodiment, a process as contemplated herein is exemplified in that the pretreatment agent (V) or pretreatment and coloring agent (VF)—based on the total weight of the pretreatment agent or pretreatment and coloring agent—has a water content of from about 15 to about 95% by weight, preferably from about 20 to about 95% by weight, more preferably from about 25 to about 95% by weight, still more preferably from about 30 to about 95% by weight and very particularly preferably from about 45 to about 95% by weight.

Particularly resistant dyeing's could be obtained when using an alkaline adjusted pretreatment agent (V) or pretreatment and dyeing agent (VF). Preferably, the pretreatment agent (V) has a pH value of about 7.0 to about 11.5, preferably from about 7.5 to about 11.0 and particularly preferably from about 8.0 to about 10.5.

In another very particularly preferred embodiment, a process as contemplated herein is exemplified in that the pretreatment agent (V) or pretreatment and coloring agent (VF) has a pH of from about 7.0 to about 11.5, preferably from about 7.5 to about 11.0 and particularly preferably from about 8.0 to about 10.5.

To adjust this alkaline pH, the pretreatment agent (V) or pretreatment and coloring agent (VF) preferably contains at least one alkalizing agent, which are added in an amount that ensures the adjustment of the optimum pH value for the respective hair treatment. The pH values for the purposes of the present disclosure are pH values measured at a temperature of about 22° C.

Depending on the choice of the desired pH value and on the presence of other components in the agent of the present disclosure, such as acidic or basic salts or buffer components, the amount of alkalizing agent added can vary, usually from about 0.01 to about 15% by weight.

As alkalizing agent, the pretreatment agent (V) or pretreatment and coloring agent (VF) may contain, for example, ammonia, alkanolamines and/or basic amino acids.

The alkanolamines which can be used in the composition of the present disclosure are preferably selected from primary amines having a C₂-C₆ alkyl base which carries at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol.

Alkanolamines particularly preferred as contemplated herein are selected from 2-aminoethan-1-ol and/or 2-amino-2-methylpropan-1-ol. A particularly preferred embodiment is therefore exemplified in that the agent as contemplated herein contains an alkanolamine selected from 2-aminoethan-1-ol and/or 2-amino-2-methylpropan-1-ol as alkalizing agent.

For the purposes of the present disclosure, an amino acid is an organic compound containing at least one protonatable amino group and at least one —COOH or —SO₃H group in its structure. Preferred amino acids are aminocarboxylic acids, especially □-(alpha)-aminocarboxylic acids and ω-aminocarboxylic acids, whereby □-aminocarboxylic acids are particularly preferred.

As contemplated herein, basic amino acids are those amino acids which have an isoelectric point pI of greater than about 7.0.

Basic □-aminocarboxylic acids contain at least one asymmetric carbon atom. In the context of the present disclosure both possible enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine, and histidine, especially preferably arginine and lysine. In another particularly preferred embodiment, an agent as contemplated herein is therefore exemplified in that the alkalizing agent is a basic amino acid from the group arginine, lysine, ornithine and/or histidine.

In addition, the product may contain other alkalizing agents, especially inorganic alkalizing agents. Inorganic alkalizing agents usable as contemplated herein are preferably selected from the group formed by sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Very particularly preferred alkalizing agents are ammonia, 2-aminoethan-1-ol (monoethanolamine), 3-aminopropan-1-ol, 4-aminobutan-1-ol, 5-aminopentan-1-ol , 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

In a further particularly preferred embodiment, a process as contemplated herein is exemplified in that the pretreatment agent (V) or pretreatment and coloring agent (VF) contains at least one alkalizing agent which is preferably selected from the group of ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Besides the alkalizing agents described above, experts are familiar with common acidifying agents for fine adjustment of the pH-value. As contemplated herein, preferred acidifiers are pleasure acids, such as citric acid, acetic acid, malic acid, or tartaric acid, as well as diluted mineral acids.

Following the application of the pretreatment agent (V) or pretreatment and coloring agent (VF), either the coloring agent (F), the posttreatment agent (N) or the posttreatment and coloring agent (NF) is applied to the keratin materials. The agents mentioned are in each case the ready-to-use agent F, N or NF.

The colorant (F), the aftertreatment agent (N) or the aftertreatment and colorant (NF) contains the film-forming polymer(s) (c) and optionally the colorant compounds (c) in a cosmetic carrier, preferably in a water-containing cosmetic carrier.

In another very particularly preferred embodiment, a process as contemplated herein is exemplified in that the colorant (F), the aftertreatment agent (N) or the aftertreatment and colorant (NF)—based on the total weight of the respective agent—has a water content of from about 15 to about 95% by weight, preferably from about 20 to about 95% by weight, more preferably from about 25 to about 95% by weight, still more preferably from about 30 to about 95% by weight and very particularly preferably from about 45 to about 95% by weight.

To produce very particularly washfast dyeing's, it has also been found to be particularly preferred if the colorant (F), the aftertreatment agent (N) or the aftertreatment and colorant (NF) is alkaline and has a pH of from about 7.0 to about 11.5, preferably from about 7.5 to about 11.0 and particularly preferably from about 8.0 to about 10.5.

In another very particularly preferred embodiment, a process as contemplated herein is exemplified in that the colorant (F), the aftertreatment agent (N) or the aftertreatment and colorant (NF) has a pH of from about 7.0 to about 11.5, preferably from about 7.5 to about 11.0 and particularly preferably from about 8.0 to about 10.5.

To adjust this alkaline pH, the colorant (F), the aftertreatment agent (N) or the aftertreatment and colorant (NF) also preferably contains at least one alkalizing agent, which are added in an amount that ensures the adjustment of the optimum pH for the respective hair treatment. The pH values for the purposes of the present disclosure are pH values measured at a temperature of about 22° C.

The colorant (F), the aftertreatment agent (N) or the aftertreatment and colorant (NF) may contain at least one alkalizing agent selected from the group of the foregoing. Particularly preferably, the colorant (F), the aftertreatment agent (N) or the aftertreatment and colorant (NF) contains at least one alkalizing agent which is preferably selected from the group of ammonia, 2-aminoethan-1-ol (monoethanolamine), 3-aminopropan-1-ol, 4-amino-butan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol , 3-amino-2-methylpropan-1-ol, 1-amino-2-methylpropan-2-ol, 3-aminopropane-1,2-diol, 2-amino-2-methylpropane-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

In another very particularly preferred embodiment, a process as contemplated herein is exemplified in that the colorant (F), the aftertreatment agent (N) or the aftertreatment and colorant (NF) contains at least one alkalizing agent which is preferably selected from the group of ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropane-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

### Procedural Steps

The technical application properties of the resulting dyeing can be further improved by selecting the optimum process conditions.

In yet another embodiment, preferred is a method comprising the following steps in the order indicated.
(1) Application of the pre-treatment agent (V) on the keratinous material,
(2) Allowing the pretreatment agent (V) to act for a period of about 10 seconds to about 10 minutes, preferably from about 10 seconds to about 5 minutes,
(3) if necessary, rinsing out the pretreatment agent (V),
(4) Application of the staining agent (F) on the keratinous material,
(5) Allow the dye (F) to react for a period of about 30 seconds to about 30 minutes, preferably from about 30 seconds to about 10 minutes, and
(6) if necessary, application of a conditioner and
(7) Rinsing of the keratinous material.

In a first step (1), the pre-treatment agent (V) is applied to the keratin materials, especially human hair.

After application, the pre-treatment agent (V) can act on the keratin materials. In this context, application times from about 10 seconds to about 10 minutes, preferably from about 20 seconds to about 5 minutes and especially preferably from about 30 seconds to about 2 minutes on the hair have proven to be particularly beneficial.

In a further preferred embodiment, a process as contemplated herein is exemplified by (2) Allow the pre-treatment agent (V) to act on the keratin materials for a period of about 10 seconds to about 10 minutes, preferably from about 10 seconds to about 5 minutes and most preferably from about 30 seconds to about 2 minutes.

In a preferred embodiment of the process as contemplated herein, the pretreatment agent (V) can now be rinsed from the keratin materials before the colorant is (F) is applied to the hair in the subsequent step.

In a further embodiment, a process comprising the following steps in the order given is particularly preferred
(1) Application of the pre-treatment agent (V) on the keratinous material,
(2) Allowing the pretreatment agent (V) to act for a period of about 10 seconds to about 10 minutes, preferably from about 10 seconds to about 5 minutes,
(3) Rinse out the pretreatment agent (V),
(4) Application of the staining agent (F) on the keratinous material,
(5) Allow the dye (F) to react for a period of about 30 seconds to about 30 minutes, preferably from about 30 seconds to about 10 minutes, and
(6) if necessary, application of a conditioner and
(7) Rinsing of the keratinous material.

Dyeing's with also good wash fastness properties were obtained when the dye (F) was applied to the keratin materials which were still exposed to the pretreatment agent (V).

In a further embodiment, a process comprising the following steps in the order given is particularly preferred
(1) Application of the pre-treatment agent (V) on the keratinous material,
(2) Allowing the pretreatment agent (V) to act for a period of about 10 seconds to about 10 minutes, preferably from about 10 seconds to about 5 minutes,
(3) no rinsing of the pre-treatment agent (V),
(4) Application of the staining agent (F) on the keratinous material,
(5) Allow the dye (F) to react for a period of about 30 seconds to about 30 minutes, preferably from about 30 seconds to about 10 minutes, and
(6) if necessary, application of a conditioner and
(7) Rinsing of the keratinous material.

In step (4) the dye (F) is now applied to the keratin materials. After application, let the colorant (F) act on the hair.

The process as contemplated herein allows the production of dyeing's with particularly good intensity and wash fastness, even with a short reaction time of the dye (F). Application times from about 10 seconds to about 10 minutes, preferably from about 20 seconds to about 5 minutes and most preferably from about 30 seconds to about 3 minutes on the hair have proven to be particularly beneficial.

In a further preferred embodiment, a process as contemplated herein is exemplified by (5) Allowing the colorant (F) to act on the hair for a period of from about 10 seconds to about 10 minutes, preferably from about 20 seconds to about 5 minutes and most preferably from about 30 seconds to about 3 minutes.

In a further embodiment, a process comprising the following steps in the order given is particularly preferred
(1) Application of the pre-treatment agent (V) on the keratinous material,
(2) Allowing the pretreatment agent (V) to act for a period of about 10 seconds to about 10 minutes, preferably from about 10 seconds to about 5 minutes,
(3) Rinse out the pretreatment agent (V),
(4) Application of the staining agent (F) on the keratinous material,
(5) Allowing the colorant (F) to act on the hair for a period of from about 10 seconds to about 10 minutes, preferably from about 20 seconds to about 5 minutes and most preferably from about 30 seconds to about 3 minutes, and
(6) if necessary, application of a conditioner and
(7) Rinsing of the keratinous material.

After exposure to the colorant (F), a conditioner can now optionally be applied.

In a further embodiment, a process comprising the following steps in the order given is particularly preferred
(1) Application of the pre-treatment agent (V) on the keratinous material,
(2) Allowing the pretreatment agent (V) to act for a period of about 10 seconds to about 10 minutes, preferably from about 10 seconds to about 5 minutes,
(3) Rinse out the pretreatment agent (V),
(4) Application of the staining agent (F) on the keratinous material,
(5) Allowing the colorant (F) to act on the hair for a period of from about 10 seconds to about 10 minutes, preferably from about 20 seconds to about 5 minutes and most preferably from about 30 seconds to about 3 minutes, and
(6) Application of a conditioner and
(7) Rinsing of the keratinous material.

In yet another embodiment, preferred is a method comprising the following steps in the order indicated.
(1) Application of the pretreatment and coloring agent (VF) on the keratinous material,
(2) Allow the pretreatment and coloring agent (VF) to act for a period of about 30 seconds to about 30 minutes, preferably from about 30 seconds to about 10 minutes,
(3) if necessary, rinsing out the pretreatment and coloring agent (VF),
(4) Application of the after-treatment agent (N) or after-treatment and coloring agent (NF) on the keratinous material,
(5) Allowing the aftertreatment agent (N) or aftertreatment and coloring agent (NF) to act for a period of from about 10 seconds to about 30 minutes, preferably from about 10 seconds to about 10 minutes, and
(6) if necessary, application of a conditioner and
(7) Rinsing of the keratinous material.

In a step (1), the pretreatment and coloring agent (VF) is first applied to the keratin materials, especially human hair.

After application, the pretreatment and coloring agent (VF) can act on the keratin materials. In this context, application times from about 30 seconds to about 30 minutes, preferably from about 30 seconds to about 15 minutes and especially preferably from about 30 seconds to about 10 minutes on the hair have proven to be particularly beneficial.

In a further preferred embodiment, a process as contemplated herein is exemplified by (2) Allowing the pretreatment and coloring agent (VF) to act on the keratin materials for a period of about 30 seconds to about 30 minutes, preferably from about 30 seconds to about 15 minutes, and most preferably from about 30 seconds to about 10 minutes.

In one embodiment of the process as contemplated herein, the pretreatment and coloring agent (VF) can now be rinsed from the keratin materials before the post-treatment agent (N), or post-treatment and coloring agent (NF) is applied to the hair in the subsequent step.

In a further embodiment, a process comprising the following steps in the order given is particularly preferred
(1) Application of the pretreatment and coloring agent (VF) on the keratinous material,
(2) Allow the pretreatment and coloring agent (VF) to act for a period of about 30 seconds to about 30 minutes, preferably from about 30 seconds to about 10 minutes,
(3) Rinse out the pretreatment and coloring agent (VF),
(4) Application of the after-treatment agent (N) or after-treatment and coloring agent (NF) on the keratinous material,
(5) Allowing the aftertreatment agent (N) or aftertreatment and coloring agent (NF) to act for a period of from about 10 seconds to about 30 minutes, preferably from about 10 seconds to about 10 minutes, and
(6) if necessary, application of a conditioner and
(7) Rinsing of the keratinous material.

Dyeing's with equally good wash fastnesses were obtained when the aftertreatment agent was (N) or post-treatment and coloring agent (NF) was applied to the keratin materials that were still exposed to the pre-treatment and coloring agent (VF).

In a further embodiment, a process comprising the following steps in the order given is particularly preferred
(1) Application of the pretreatment and coloring agent (VF) on the keratinous material,
(2) Allow the pretreatment and coloring agent (VF) to act for a period of about 30 seconds to about 30 minutes, preferably from about 30 seconds to about 10 minutes,
(3) no rinsing of the pretreatment and coloring agent (VF),
(4) Application of the after-treatment agent (N) or after-treatment and coloring agent (NF) on the keratinous material,
(5) Allowing the aftertreatment agent (N) or aftertreatment and coloring agent (NF) to act for a period of from about 10 seconds to about 30 minutes, preferably from about 10 seconds to about 10 minutes, and
(6) if necessary, application of a conditioner and
(7) Rinsing of the keratinous material.

In step (4), the post-treatment agent (N) or post-treatment and coloring agent (NF) is now applied to the keratin materials. After application, the after-treatment agent (N) or after-treatment and coloring agent (NF) is now left to act on the hair.

Even with a short reaction time of the aftertreatment agent, the process as contemplated herein permits (N) or aftertreatment and dyeing agent (NF) the production of dyeing's with particularly good intensity and wash fastness. Exposure times of about 10 seconds to about 30 minutes or up to about 20 minutes or up to about 10 minutes, preferably from about 20 seconds to about 5 minutes and most preferably from about 30 seconds to about 3 minutes on the hair have proven to be particularly advantageous.

In a further preferred embodiment, a process as contemplated herein is exemplified by (5) Allowing the aftertreatment agent (N) or aftertreatment and coloring agent (NF) to act on the hair for a period of from about 10 seconds to about 30 minutes, preferably from about 20 seconds to about 5 minutes, and most preferably from about 30 seconds to about 3 minutes.

In a further embodiment, a process comprising the following steps in the order given is particularly preferred
(1) Application of the pretreatment and coloring agent (VF) on the keratinous material,
(2) Allow the pretreatment and coloring agent (VF) to act for a period of 30 seconds to 30 minutes, preferably from about 30 seconds to about 10 minutes,
(3) Rinse out the pretreatment and coloring agent (VF),
(4) Application of the after-treatment agent (N) or after-treatment and coloring agent (NF) on the keratinous material,
(5) Allowing the after-treatment agent (N) or after-treatment and coloring agent (NF) to act on the hair for a period of from about 10 seconds to about 30 minutes, preferably from about 20 seconds to about 5 minutes, and most preferably from about 30 seconds to about 3 minutes, and
(6) application of a conditioner, if necessary, and
(7) Rinsing of the keratinous material.

After the post-treatment agent (N) or post-treatment and coloring agent (NF) has taken effect, a conditioner can now optionally be applied.

In a further embodiment, a process comprising the following steps in the order given is particularly preferred
(1) Application of the pretreatment and coloring agent (VF) on the keratinous material,
(2) Allow the pretreatment and coloring agent (VF) to act for a period of about 30 seconds to about 30 minutes, preferably from about 30 seconds to about 10 minutes,
(3) Rinse out the pretreatment and coloring agent (VF),
(4) Application of the after-treatment agent (N) or after-treatment and coloring agent (NF) on the keratinous material,
(5) Allowing the after-treatment agent (N) or after-treatment and coloring agent (NF) to act on the hair for a period of from about 10 seconds to about 30 minutes, preferably from about 20 seconds to about 5 minutes, and most preferably from about 30 seconds to about 3 minutes, and
(6) Application of a conditioner and
(7) Rinsing of the keratinous material.

Preferably, the conditioner contains at least one cationic and/or non-ionic surfactant.

Surprisingly, it turned out that using the conditioner—especially if it contains at least one cationic surfactant—may further improve the fastness of the dyeing' s obtained and further intensify the color result.

In a further preferred embodiment, a process as contemplated herein is exemplified in that the conditioner contains at least one cationic and/or non-ionic surfactant.

In another particularly preferred version, a process as contemplated herein is exemplified in that the conditioner contains at least one cationic surfactant.

To obtain as homogeneous and resistant a coloration as possible, it has been found particularly preferable if a period of maximum about 48 hours, preferably maximum about 24 hours, further preferred maximum about 12 hours and very particularly preferred maximum about 6 hours lies between the application of the pretreatment agent (V) or the pretreatment and colorant (VF) and the application of the colorant (F), the aftertreatment agent (N) or the aftertreatment and colorant (NF).

In a further preferred embodiment, a method as contemplated herein is exemplified in that the pretreatment agent (V) or the pretreatment and coloring agent (VF) and the coloring agent (F) or the posttreatment agent (N) or the posttreatment and coloring agent (NF) are applied to the hair within a period of not more than about 48 hours, preferably not more than about 24 hours, more preferably not more than about 12 hours and very particularly preferably not more than about 6 hours.

### Manufacturing Process

A fourth object of the present disclosure is a process for preparing a cosmetic agent for use in coloring keratinous material, in particular human hair, containing at least one organic silicon compound (a), comprising the following steps in the order indicated:
(1) Providing at least one amino silane (a1) of formula (I),

R₁R₂N-L-Si(OR₃)ₐ(R₄)₃₋ₐ (I),

where

R₁, R₂ independently represent a hydrogen atom, a C₁-C₆ alkyl group, a hydroxy-C₁-C₆ alkyl group, a C₂-C₆ alkenyl group, an amino-C₁-C₆ alkyl group, an amino-C₁-C₆ C₁-C₆ alkyl group or a group of formula (III),

-(L′)_{c}-Si(R₆)_{b}(OR₅)_{3-b} (III),

where

L and L′ each independently represent a linear or branched C₁-C₂₀ divalent alkylene group, preferably a linear C₁-C₂ or C₃ alkylene group,

R₃ and R₅ independently represent a C₁-C₆ alkyl group, preferably a C₁-C₂ alkyl group,

R₄ and R₆ independently represent a C₁-C₆ alkyl or a C₂-C₆ alkenyl group,

a and b each independently represent an integer from about 2 to about 3, preferably about 3, and

Is c 0 or 1;

and optionally at least one second silane (a2) of formula (II)

R₇—Si(OR₈)_{d}(R₉)_{3-d} (II),

where

R₇ represents a linear or branched C₁-C₁₂ alkyl group, hydroxy-C₁-C₁₂ alkyl group or C₂-C₁₂ alkenyl group, preferably a linear C₁-C₆ alkyl or C₂-C₆ alkenyl group, more preferably a C₁-C₂ alkyl group,

R₈ is a C₁-C₆ alkyl group, preferably a C₁-C₂ alkyl group,

R₉ is a C₁-C₆ alkyl or a C₂-C₆ alkenyl group,

d stands for an integer from about 2 to about 3;
(2) partial hydrolysis and condensation of the silanes presented in step (1) by addition of a substoichiometric amount of water, preferably with removal of the liberated alcohols by distillation at reduced pressure.

In various embodiments in which amino silanes (a1) of the formula (I) and silanes (a2) of the formula (II) are used, the mass ratio of all compounds of the formula (I) to all compounds of the formula (II) is preferably from about 5:1 to about 1:20, more preferably from about 1:1 to about 1:10, still more preferably from about 1:2 to about 1:5.

In various embodiments in which amino silanes (a1) of the formula (I) and silanes (a2) of the formula (II) are used, the molar ratio of all compounds of the formula (I) to all compounds of the formula (II) is preferably from about 2:1 to about 1:30, more preferably from about 1:1 to about 1:20, still more preferably from about 1:2 to about 1:10.

The water is used in a substoichiometric amount, i.e., an amount less than that which would theoretically be required to hydrolyze all the hydrolysable groups present on the Si atoms, i.e., the alkoxysilane groups (hence “partial hydrolysis”). Preferably, the amount of water used for this purpose is at least 10% below the amount stoichiometrically required for complete hydrolysis, and preferably at least 20% below. Particularly preferably, the amount of water used for hydrolysis is about 0.2 to about 2.5 moles of water per 1 mole of Si, preferably from about 0.4 to about 2.0 moles of water per 1 mole of Si, more preferably from about 0.6 to about 1.2 moles of water per 1 mole of Si. During hydrolysis, the hydrolysable groups are attached to the Si-atoms are hydrolyzed and, since they are alkoxy groups, the corresponding alcohols are split off. Si—OH groups remain on the silicon, which can react with each other in the next step in a condensation reaction, i.e., with water splitting off. The amount of water is such that the condensation is a partial condensation, where “partial condensation” or “partial condensation” in this context means that not all the condensable groups of the silanes presented react with one another, so that the resulting organic silicon compound (a) still has on average at least one hydrolysable/condensable group per molecule in each case. Preferably, the average number of condensable/hydrolysable groups per molecule of compound (a) is at least about 1.5, more preferably at least about 2.

The water can be added continuously, in partial quantities or directly as a total quantity. The addition is preferably carried out under stirring. To ensure temperature control, the reaction mixture can be cooled, or the amount and rate of water added can be adjusted. Depending on the amount of silanes used, the addition and reaction can take place over a period of about 2 minutes to about 72 hours. Preferably, the addition is continuous, especially on an industrial scale.

It may be preferred in various embodiments that the temperature during hydrolysis in step (2) does not exceed 75° C., preferably 60° C. Preferably, the temperature during the hydrolysis reaction and optionally also during the condensation reaction is in the range of about 10 to about 75° C., preferably from about 20 to about 60° C.

It is further preferred that the hydrolysis reaction takes place under inert gas, such as nitrogen, or it is otherwise ensured that the reaction mixture does not meet additional moisture, such as humidity. The reaction therefore preferably takes place in a reaction vessel that is closed to the ambient atmosphere, i.e., with the exclusion of moisture.

The hydrolysis reaction is followed by a condensation reaction in which the Si—O—Si bonds are formed. To shift the equilibrium of the condensation reaction in the direction of the products, it is provided as contemplated herein that the condensation reaction (partial condensation) is carried out under reduced pressure to remove the resulting alcohols and possibly also water from the reaction mixture and transfer them to the gas phase. This suppresses the back reaction and shifts the equilibrium of the reaction to the side of the condensates. Preferably, the reduced pressure is achieved by vacuum distillation, in which the reaction mixture is subjected to reduced pressure, typically to a maximum of about 800 mbar, preferably to a maximum of about 500 mbar, for example about 50-800 or about 50-500 mbar, and the volatile alcohols and optionally also water are condensed and collected as liquid distillate in a receiver. Distillation can optionally be carried out with cooling of the evaporated alcohols/water by employing a cooler. The reduced pressure can be generated by employing common processes known in the prior art, typically with a vacuum pump.

In various embodiments of the present disclosure, silanes (a1) and (a2) are predominantly, i.e., at least about 50% by weight, preferably at least about 75% by weight, almost exclusively, i.e., at least about 90% by weight, preferably about 95% by weight, or exclusively those which carry methoxy silane or ethoxy silane groups, di- and trimethoxy- and ethoxy silanes, particularly preferably trimethoxy- or triethoxysilane. These have the advantage that methanol and ethanol are released during hydrolysis and condensation, respectively, which can be easily removed from the reaction mixture by vacuum distillation due to their boiling points.

In various embodiments of the present disclosure, the preparation is carried out in a two- or multi-step process in which, in a first step, hydrolysis is carried out by adding sub-stoichiometric amounts of water, either continuously, stepwise or in one, under normal pressure. Only after the water has been added, preferably completely, is reduced pressure applied in a subsequent step and the resulting/emerging alcohols removed by vacuum distillation. In this embodiment, vacuum distillation preferably occurs after at least about 50% by weight of the total designed amount of water has been added, preferably at least about 70%, about 80%, about 90%, about 95% or about 100% by weight of the water. In such embodiments, step (2) can thus be divided into a first step (2a), in which the water is added, and a step (2b), in which vacuum distillation takes place.

Alternatively, in various embodiments of the present disclosure, vacuum distillation may be performed simultaneously with hydrolysis. In such embodiments, the pressure is already reduced before the water is added, at the start of the addition, or after about 5-20 wt % of the total planned amount of water has been added.

In various embodiments, vacuum distillation is carried out under conditions that yield a product containing less than about 5 wt %, preferably less than about 2 wt %, more preferably less than about 1 wt % free alcohols (from the hydrolysis reaction). The water content of the product after vacuum distillation is less than about 1% by weight, even more preferably less than about 0.1% by weight, and most preferably less than about 0.01% by weight.

The condensation reaction at reduced pressure can be carried out at elevated temperature. For this purpose, the reaction mixture can be actively heated. In various embodiments, the temperature can be adjusted such that the alcohols released during the condensation reaction can be vaporized and removed at the applied reduced pressure. In various preferred embodiments, however, the temperature is no more than about 75° C., preferably no more than about 60° C. It may further be preferred that there is no active heating of the reaction mixture and that any increase in temperature above ambient temperature is caused only by the exotherm of the hydrolysis.

In various embodiments of the present disclosure, the reaction may also be carried out in the presence of solvents, i.e., alcohols such as methanol or ethanol. These are then usually used in about 0.1 to about 5 times the amount by weight based on the silanes used and then removed by distillation.

It has been found to be quite suitable to use monomeric aminotrialkoxysilanes (a1) with one silane group, i.e., one silicon atom per molecule, and monomeric trialkoxysilanes (a2). For example, it may be preferred to use aminopropyltri(m)ethoxy silane as component (a1) in combination with an alkyltrialkoxysilane, for example alkyltri(m)ethoxy silane, especially C₁₋₃ alkyltri(m)ethoxy silane as component (a2), to obtain the organic silicon compound (a).

In various embodiments, the quotient of the molar ratio of Si/hydrolysable group (especially alkoxy group) in the produced molecule/oligomer is at least about 0.3, preferably at least about 0.5, more preferably at least about 0.7, for example at least about 1.

Organic silicon compounds a) as contemplated herein have, for example, a hydrolysable group/alkoxy group content of, for example, about 5 to about 30% by weight.

Organic silicon compounds a) as contemplated herein can be chain or cyclic. They are mixtures of chain and/or cyclic oligomers.

The degree of oligomerization is typically about 2 to about 30, i.e., the organic silicon compounds a) as contemplated herein include about 2 to about 30 monomeric units derived from the compounds a1 and, if used, also a2, preferably from about 3 to about 20 units.

About the further preferred embodiments of the multicomponent packaging unit as contemplated herein and the methods as contemplated herein, what has been said about the agents as contemplated herein applies mutatis mutantis.

### EXAMPLES

### 1. Formulations

The following formulations were prepared (unless otherwise stated, all figures are in % by weight)

Pretreatment Agent (V)

| Agent (I) | (I) |
|---|---|
| Mixture containing partial condensate of (3- | 1.5 g |
| Aminopropyl)triethoxysilane (AMEO) and | |
| methyltrimethoxysilane (MTMS) | |

The partial condensate was obtained by adding 700.5 g of MTMS and 199.5 g of AMEO and mixing with stirring in a closed reaction vessel. Then 100.5 g of water was added slowly with stirring and the mixture was further stirred for 20 minutes. Subsequently, the resulting alcohols were distilled off for 60 minutes under vacuum at 50° C. and the remaining condensate was stabilized by adding 499.5 g of volatile silicone (Xiameter PMX 200; Dow Chemical).

| Agent (II) | (II) | |
|---|---|---|
| Sodium hydroxide (Natrosol 250 M) | 0.2 | g |
| Sodium methylparaben | 0.08 | g |
| Pigment in PEG-12 Dimethicone | 1.5 | g |
| (Xiameter OFX-0193) | | |
| Water | Ad 21.5 | g |

By mixing 1.5 g of agent (I) and 21.5 g of agent (II), the ready-to-use pretreatment and coloring agent (VF) was prepared. This involved shaking agents (I) and (II) together for 3 minutes. Then the pretreatment and colorant (VF) could stand for about 5 minutes. The pH value of the ready-to-use pretreatment and colorant (VF) was about 10.

As a comparison, the same agent was prepared without the use of vacuum in the preparation of the partial condensate.

2. Application

One strand of hair (Kerling, Euronatur hair white) was dipped into the pretreatment and dyeing medium (VF) and left for 1 minute. After that, superfluous agent was stripped from each strand of hair. Each strand of hair was washed out with water. Excess water was scraped off each strand of hair.

Then, the hair strands were each washed and dried 20 times with a small amount of shampoo (handle) (0.3 g foam shampoo 7-herbs per 1 g hair>shampooed for 45 sec>rinsed, dried). Subsequently, the strands were rated visually (1=best durability, 6=worst durability).

| | | Durability assessment | |
|---|---|---|---|
| Resting time | | (after 20 hair washes*) | |
| after | | Production | Production |
| coloration | | WITHOUT vacuum | WITH vacuum |
| Immediately | | 5 | 5 |
| after coloration | | | |
| 1 | h | 5 | 4 |
| 2 | h | 5 | 3 |
| 6 | h | 4 | 2 |
| 24 | h | 3 | 2 |
| 48 | h | 2 | 2 |

The coloration with agent (I), which was prepared under vacuum, leads to a significantly better durability of the coloration.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A composition for dyeing keratinous material, especially human hair, comprising in a cosmetic carrier
(a) at least one organic silicon compound obtainable by partial condensation under reduced pressure of at least one amino silane (a1) of the formula (I),
R₁R₂N-L-Si(OR₃)ₐ(R₄)₃₋ₐ (I),
wherein
R₁, R₂ independently represent a hydrogen atom, a C₁-C₆ alkyl group, a hydroxy-C₁-C₆ alkyl group, a C₂-C₆ alkenyl group, an amino-C₁-C₆ alkyl group, an amino-C₁-C₆ alkyl-amino-C₁-C₆ alkyl group or a group of formula (III),
-(L′)_{c}-Si(R₆)_{b}(OR₅)_{3-b} (III),
wherein
L and L′ each independently represent a linear or branched C₁-C₂₀ divalent alkylene group,
R₃ and R₅ independently represent a hydrogen atom or a C₁-C₆ alkyl group,
R₄ and R₆ independently represent a C₁-C₆ alkyl or a C₂-C₆ alkenyl group,
a and b each independently represent an integer from 2 to 3, and
c is 0 or 1;
and optionally at least one second silane (a2) of formula (II)
R₇—Si(OR₈)_{d}(R₉)_{3-d} (II),
wherein
R₇ represents a linear or branched C₁-C₁₂ alkyl group, hydroxy-C₁-C₁₂ alkyl group or C₂-C₁₂ alkenyl group,
R₈ represents a hydrogen atom or a C₁-C₆ alkyl group,
R₉— is a C₁-C₆ alkyl or a C₂-C₆ alkenyl group,
d is an integer from 2 to 3; and
(b) at least one colorant compound.

2. The composition according to claim 1, wherein the partial condensation comprises: (a) hydrolysis by addition of substoichiometric amounts of water, and (b) distillation at reduced pressure of the alcohols released during hydrolysis.

3. The composition according to claim 1, wherein the partial condensation takes place at reduced pressure in the range from about 50 to about 800 mbar.

4. The composition according to claim 1, wherein the at least one amino silane (a1) comprises a compound of formula (I) wherein
(i) R₁, R₂ both represent a hydrogen atom, a methyl or ethyl group; and/or
(ii) R₁ represents a hydrogen atom, a C₁-C₆ alkyl group, a hydroxy-C₁-C₆ alkyl group, a C₂-C₆ alkenyl group or an amino-C₁-C₆ alkyl group, and R₂ represents a grouping of formula (III) wherein b, L′, R₅ and R₆ are preferably identical to a, L, R³ and R⁴.

5. The composition according to claim 1, wherein R₃ is a methyl group or ethyl group and a is 3.

6. The composition according to claim 1, wherein the at least one amino silane (a1) of formula (I) is selected from the group of
(3-aminopropyl)triethoxysilane
(3-aminopropyl)trimethoxysilane
1-(3-aminopropyl)silanetriol
(2-aminoethyl) triethoxysilane
(2-aminoethyl) trimethoxysilane
-1-(2-aminoethyl) silanetriol,
Aminomethyltrimethoxysilane
Aminomethyltriethoxysilane
Aminomethylsilanetriol
N-(2-aminoethyl)-3-Aminopropyltrimethoxysilane
N-(2-aminoethyl)-3-Aminopropyltriethoxysilane
N-(2-aminoethyl)-3-Aminopropylsilanetriol
(3-Dimethylaminopropyl)triethoxysilane
(3-Dimethylaminopropyl)trimethoxysilane
1-(3-Dimethylaminopropyl)silanetriol
(2-Dimethylaminoethyl)triethoxysilane
(2-Dimethylaminoethyl)trimethoxysilane
-1-(2-Dimethylaminoethyl)silanetriol,
Dimethylaminomethyltrimethoxysilane
Dimethylaminomethyltriethoxysilane
Dimethylaminomethylsilanetriol
-(3-Diethylaminopropyl)triethoxysilane
(3-Dimethylaminopropyl)trimethoxysilane
1-(3-Diethylaminopropyl)silanetriol
(2-diethylaminoethyl)triethoxysilane
(2-diethylaminoethyl)trimethoxysilane
-1-(2-diethylaminoethyl)silanetriol,
Diethylaminomethyltrimethoxysilane
Diethylaminomethyltriethoxysilane
Diethylaminomethylsilanetriol
3-(Trimethoxysilyl)-N-[3-(trimethoxysilyl) propyl]-1-propanamine
3-(Triethoxysilyl)-N-[3-(triethoxysilyl) propyl]-1-propanamine
N-methyl-3-(Trimethoxysilyl)-N-[3-(trimethoxysilyl) propyl]-1-propanamine
N-methyl-3-(Triethoxysilyl)-N-[3-(triethoxysilyl) propyl]-1-propanamine
2-[bis[3-(Trimethoxysilyl) propyl] amino] ethanol
2-[bis[3-(Triethoxysilyl) propyl] amino] ethanol
3-(Trimethoxysilyl)-N,N-bis[3-(trimethoxysilyl) propyl]-1-propanamine
3-(Triethoxysilyl)-N,N-bis[3-(triethoxysilyl)propyl]-1-propanamine
N1,N1-Bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl) propyl]-2-propene-1-amine, or
N,N-bis[3-(triethoxysilyl)propyl]-2-propen-1-amine.

6. The composition according to claim 1, wherein the at least one silane (a2) comprises a compound of formula (II), wherein d is 3 and R₇ and R₈ are each methyl or ethyl.

7. The composition according to claim 6, wherein the at least one silane (a2) is selected from the group of:
Methyltrimethoxysilane; Ethyltrimethoxysilane; Methyltriethoxysilane; Ethyltriethoxysilane; Dimethyldimethoxysilane; Diethyldiethoxysilane; Dimethyldiethoxysilane; Diethyldimethoxysilane; Vinyltrimethoxysilane; Vinyltriethoxysilane; Tetraethylorthosilicate; or Tetramethylorthosilicate.

8. The composition according to claim 1, wherein the composition further comprises (d) at least one compound selected from the group of silicones.

9. The composition according to claim 8, wherein the at least one compound selected from the group of silicones (d) is selected from siloxanes of formula (IV)
wherein
(R₁₀)₃Si—O—(Si(R₁₀)₂—O)ₖ—Si(R₁₀)₃ (IV),
each R₁₀ independently represents a C₁-C₆ alkyl group,
k is 0 or an integer from about 1 to about 30.

10. The composition according to claim 1, wherein the composition comprises as compound (b):
(i) at least one coloring compound selected from the group of inorganic and/or organic pigments; and/or
(ii) at least one anionic, cationic and/or nonionic direct dye.

11. The composition according to claim 1, wherein the composition comprises at least one film-forming polymer (c).

12. A multicomponent packaging unit (kit-of-parts) for dyeing keratinous material, in particular human hair, which comprises
a first container comprising a cosmetic product (I) and
a second container comprising a cosmetic product (II) and
a third container comprising a cosmetic product (III), wherein
the agent (I) comprises at least one organic silicon compound (a) as defined in claim 1 and optionally also a silicone (d) as defined in claim 1,
the composition (II) comprises water and optionally at least one colorant compound (b) as defined in claim 1, and
the agent (III) comprises at least one film-forming polymer (c) as defined in claim 1 and optionally at least one color-imparting compound (b) as defined in claim 1,
wherein either the agent (II), the agent (III) or both contain at least one color-imparting compound (b).

13. A method for dyeing keratinous material, in particular human hair, comprising the following steps in the order indicated:
(A)
(1) Application of a pretreatment agent (V) to the keratinous material, wherein the pretreatment agent (V) comprises, in a water containing cosmetic carrier, at least one organic silicon compound (a) as defined in claim 1 and optionally also a silicone (d) as defined in claim 8, and
(2) application of a colorant (F) to the keratinous material, the colorant comprising at least one color-imparting compound (b) as defined in claim 1 and at least one film-forming polymer (c) as defined in claim 1; or
(B)
(1) Application of a pretreatment and coloring agent (VF) to the keratinous material, wherein the pretreatment and coloring agent (VF) comprises, in a water containing cosmetic carrier, at least one organic silicon compound (a) as defined in claim 1, at least one coloring compound (b) as defined in claim 1, and optionally also a silicone (d) as defined in claim 8, and
(2) Application of an aftertreatment agent (N) to the keratinous material, wherein the aftertreatment agent comprises at least one film-forming polymer (c) as defined in claim 1;
or (C)
(1) Application of a pretreatment and coloring agent (VF) to the keratinous material, wherein the pretreatment and coloring agent (VF) comprises, in a water containing cosmetic carrier, at least one organic silicon compound (a) as defined in claim 1, at least one coloring compound (b) as defined in claim 1, and optionally also a silicone (d) as defined in claim 8, and
(2) Application of a post-treatment and coloring agent (NF) to the keratinous material, wherein the post-treatment and coloring agent comprises at least one coloring compound (b) as defined in claim 1 and at least one film-forming polymer (c) as defined in claim 1.

14. The method according to claim 13, wherein the pretreatment agent (V) or pretreatment and coloring agent (VF) is prepared by mixing a first agent (I) and a second agent (II) prior to application to the keratinous material, the first agent (I) and the second agent (II) being as defined in claim 1.

15. The method according to claim 13, wherein the pretreatment agent (V) or pretreatment and coloring agent (VF)—based on the total weight of the agent—has a water content of from about 15 to about 95% by weight.

16. The method of claim 13, further comprising the step of applying a conditioner after the steps of pretreatment, coloring, and optionally posttreatment.

17. The method according to claim 16, wherein the conditioner comprises at least one cationic and/or nonionic surfactant, preferably a polydimethylsiloxane, more preferably a cationically modified polydimethylsiloxane, most preferably a polydimethylsiloxane having one or more terminal aminoalkyl group(s).

18. The composition according to claim 1, wherein at least one of R₁ and R₂ represent a group of formula (III), and wherein a and b each independently represent 3.

19. The composition according to claim 1, wherein the at least one colorant compound (b) is selected from the group of photochromic dyes, thermochromic dyes, pigments, mica, or direct dyes.

20. The composition according to claim 11, wherein the at least one film-forming polymer (c) is selected from the group of poly(meth)acrylic acid polymers.
